SEQUENCE LISTING (2 sequences)

SEQ ID NO: 1 - protein, 322 aa, Homo sapiens
MDPTISTLDTELTPINGTEETLCYKQTLSLTVLTCIVSLVGLTGNAVVLWLLGCRMRRNAFSIYILNLAAADFLFLSGRLIYSLLSFISIPHTISKILYPVMMFSYFAGLSFLSAVSTERCLSVLWPIWYRCHRPTHLSAVVCVLLWALSLLRSILEWMLCGFLFSGADSAWCQTSDFITVAWLIFLCVVLCGSSLVLLIRILCGSRKIPLTRLYVTILLTVLVFLLCGLPFGIQFFLFLWIHVDREVLFCHVHLVSIFLSALNSSANPIIYFFVGSFRQRQNRQNLKLVLQRALQDASEVDEGGGQLPEEILELSGSRLEQ

SEQ ID NO: 2 - protein, 23 aa, Homo sapiens
GIGCVWHWKHRVATRFTLPRFLQ

(19) United States
(12) Patent Application Publication
(10) Pub. No.: US 2011/0150769 A1
(43) Pub. Date: Jun. 23, 2011

(54) IDENTIFICATION AND USE OF COMPOUNDS FOR TREATING PERSISTENT PAIN

(76) Inventors: David J. Anderson, Altadena, CA (US); Xinzhong Dong, Clarksville, MD (US); Qin Liu, Baltimore, MD (US); Yun Guan, Woodstock, MD (US)

(21) Appl. No.: 12/851,436

(22) Filed: Aug. 5, 2010

Related U.S. Application Data
(60) Provisional application No. 61/231,922, filed on Aug. 6, 2009.

(51) Int. Cl. A61K 49/00; A61K 31/7088; A61K 38/02; A61K 31/501; A61K 31/5517; A61P 25/04; A61P 29/00

(52) U.S. Cl. 424/9.2; 514/44 R; 514/18.3; 514/252.02; 514/219

(57) ABSTRACT

The present application provides methods and compositions that can be used to treat persistent pain and to identify compounds that can be used for treating persistent pain. More specifically, agonists of members of the Mrgpr receptor family, particularly agonists of MrgprX1, can identified and screened for use in treating persistent pain, such as pain caused by inflammation or nerve injury.

### RELATED APPLICATIONS

The present application claims priority under 35 U.S.C. §119(e) to U.S. Provisional Application No. 61/231,922, filed on Aug. 6, 2009, which is herein expressly incorporated by reference in its entirety.

### STATEMENT REGARDING FEDERALLY SPONSORED R&D

This invention was made with government support under Grant Numbers NS054791, NS58481, NS26363, and NS048499 awarded by the National Institutes of Health. The government has certain rights in the invention.

### REFERENCE TO SEQUENCE LISTING

The present application is being filed along with a Sequence Listing in electronic format. The Sequence Listing is provided as a file entitled CALTE_{—}066_SEQLIST.TXT, created Aug. 5, 2010, which is 4 kb in size. The information in the electronic format of the Sequence Listing is incorporated herein by reference in its entirety.

### BACKGROUND

1. Field of the Application

The present application relates generally to the field of pain management. More particularly, the application relates to treatment and prevention of persistent pathological pain states, such as inflammatory and neuropathic pain.

2. Description of the Related Art

Pain is experienced both acutely, and chronically. If uncontrolled, persistent (or chronic) pain, such as persistent pain caused by inflammation or nerve injury may lead to various unremitting pain symptoms (e.g., tactile allodynia, thermal hyperalgesia, spontaneous pain) in patients. Patients with such persistent pain states have few treatment options, in part because pain-specific drug targets are lacking.

The therapeutic potentials of glutamate NMDA receptors, which play roles in the establishment of central sensitization, and mu-opioid receptor, which acts to suppress central sensitization and alleviate inflammatory pain, have been explored. However, the therapeutic potentials of these molecules for persistent pain are often compromised because of their widespread expression in the brain. Therefore, there is a great need for new pain-specific targets for pain management, as well as new and pain-specific therapeutic agents for the treatment of persistent pain, such as inflammatory pain and neuropathic pain.

### SUMMARY OF SOME EMBODIMENTS

In some aspects, the present application provides methods of identifying a compound for reducing persistent pain in a subject, where the methods generally comprise providing and/or identifying one or more MrgprX1 agonists and testing the MrgprX1 agonists for their ability to reduce or inhibit persistent pain. For example, they may be tested in an animal model of persistent pain. MrgprX1 agonists that are able to reduce or inhibit persistent pain are selected.

In some embodiments, in addition to testing for the ability to reduce or inhibit persistent pain, the MrgprX1 agonists are tested for their ability to modulate (increase or decrease) the perception of acute pain. For example, they may be tested for their activity in an animal model of acute pain. MrgprX1 agonists are selected that reduce or inhibit persistent pain but do not alter perception of acute pain. In some embodiments, MrgprX1 agonists that do not reduce or inhibit persistent pain and/or do modulate acute pain are eliminated.

In some embodiments, one or more of the MrgprX1 agonists reduces or inhibits persistent pain by directly activating MrgprX1 receptors. In some embodiments, the MrgprX1 agonist reduces or inhibits persistent pain by positively allosterically modulating a ligand of MrgprX1. Non-limiting examples of agonists of MrgprX1 include BAM8-22, and P60 peptide.

In some embodiments, the MrgprX1 agonist is selected from the group consisting of small molecules, peptides, and nucleic acids. In some embodiments, the MrgprX1 agonist can be a small molecule. In some embodiments, the MrgprX1 agonist can be a peptide. In some embodiments, the MrgprX1 agonist can be a nucleic acid.

In some embodiments, testing the MrgprX1 agonists for their ability to reduce or inhibit persistent pain in an animal model of persistent pain comprises administering the MrgprX1 agonists directly into the spinal cord of an animal in the animal model. In some embodiments, testing the MrgprX1 agonist comprises determining the effect of the MrgprX1 agonist on responses to painful stimuli in an animal in an inflammatory state.

In some aspects, the present application provides methods of treating or preventing persistent pain in a subject in need thereof, where the methods generally comprise identifying a subject suffering from or at the risk of developing persistent pain and administering to the subject an effective amount of an MrgprX1 agonist. The methods may also comprise the step of identifying MrgX1 agonists that reduce persistent pain but do not modulate acute pain, for example, in animal models of persistent and acute pain. In some embodiments, the persistent pain is caused by inflammation. In some embodiments, the persistent pain is caused by nerve injury. In some embodiments, the MrgprX1 agonist binds to MrgprX1. In some embodiments, the MrgprX1 agonist is a ligand of MrgprX1. In some embodiments, the MrgprX1 agonist is a positive allosteric modulator of a ligand of MrgprX1. Non-limiting examples of agonists of MrgprX1 include Bovine adrenal medulla 22 (BAM 22), BAM8-22, and P60 peptide comprising an amino acid sequence of SEQ ID NO:2.

In some embodiments, the MrgprX1 agonist activates MrgprX1 by enhancing the activation activity of a second MrgprX1 agonist. In some embodiments, the MrgprX1 agonist is selected from the group consisting of small molecules, peptides and nucleic acids. In some embodiments, the MrgprX1 agonist can be a small molecule. Non-limiting examples of small molecule MrgprX1 agonists include N-[3-(5-Chloro-6-oxo-4-piperazin-1-yl-6H-pyridazin-1-ylmethyl)-2-methyl-phenyl]-4-(6-methoxy-pyridin-3-yl)-benzamide, N-[3-(6-oxo-4-Piperazin-1-yl-6H-pyridazin-1-ylmethyl)-2-methylphenyl]-4-(6-methoxy-pyridin-3-yl)-benzamide, and

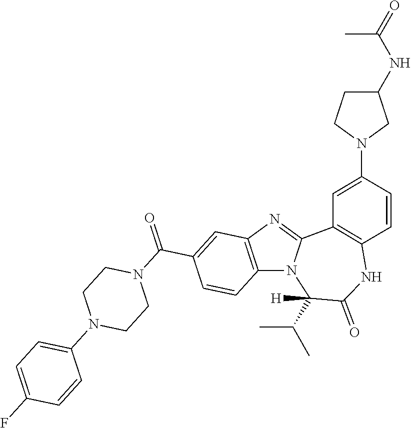

In some embodiments, the MrgprX1 agonist can be a peptide. Non-limiting examples of peptide MrgprX1 agonist include BAM 22, BAM 8-22, BAM 1-7, and P60 peptide comprising an amino acid sequence of SEQ ID NO:2.

In some embodiments, the subject is a mammal. In some embodiments, the mammal is human.

In some embodiments, the MrgprX1 agonist is delivered directly into the spinal cord of the subject.

In other aspects, the present application provides a pharmaceutical composition for the treatment of prevention of persistent pain, where the pharmaceutical composition comprises an effective amount of an MrgprX1 agonist. In some embodiments, the MrgprX1 agonist can be a small molecule. In some embodiments, the MrgprX1 agonist can be a peptide. In some embodiments, the MrgprX1 agonist can be a nucleic acid.

### BRIEF DESCRIPTION OF THE DRAWINGS

FIGS. 1A-C show the targeted deletion of a cluster of 12 intact Mrgpr coding sequences. FIG. 1A is a schematic diagram showing the Mrgpr gene cluster on wild-type mouse chromosome 7. The distance between MrgprA1 and MrgprB4 is 845 kilobases, which contains 12 intact Mrgprs (represented by each black block with its name on top). Targeting constructs, containing loxP sites (black triangles) and the selection marker genes, were introduced to the MrgprA1 and MrgprB4 loci in ES cells by two rounds (1^{st} and 2^{nd}) of electroporation and homologous recombination. Positive ES clones with correct targeting in the two loci underwent a third round of electroporation with CMV-Cre construct. Cre-mediated recombination resulted in deletion of an Mrgpr cluster between loxP sites. The deletion event in ES cells (lane 1 and 2; lane 3 as negative control using wild-type ES cells) was detected by PCR amplification using primers 1 and 2 flanking the cluster (shown as small arrows). The PCR product (456 base pairs) was further confirmed by sequencing. FIG. 1B is a Southern blot of genomic DNA with an MrgprC or MrgprA probe. The genomic DNA was digested with BgIII Due to cross-hybridization, a single MrgprC or MrgprA probe can label multiple members of the MrgprC or MrgprA subfamily in WT (+/+) and cluster heterozygous mice (+/−) DNA. In homozygous mice (−/−), most of the positive bands are absent (arrows). FIG. 1C is a table showing that the deletion of Mrgpr genes does not affect the cell fate determination of small-diameter sensory neurons. The proportion of nonpeptidergic (IB4⁺) and peptidergic (CGRP⁺) small-diameter sensory neurons does not differ between WT and Mrgpr-clusterΔ^{-/-} mice (KO mice, n=3).

FIGS. 2A-J show the enhanced inflammatory pain responses in Mrgpr-clusterΔ^{-/-} (KO) mice. FIGS. 2A-C are histograms showing that Mrgpr-clusterΔ^{-/-} mice responded normally to noxious acute thermal stimuli. Response latencies in tail immersion (50° C., n=12, FIG. 2A), hot plate (50° C., n=11, FIG. 2B) and Hargreaves (n=24, FIG. 2C) tests did not differ between WT and Mrgpr-clusterΔ^{-/-} mice. FIG. 2D is a graph showing that the paw withdrawal threshold of Mrgpr-clusterΔ^{-/-} mice to punctate mechanical stimuli (Von Frey) was comparable to that of WT mice (n=12). FIG. 2E is a histogram showing that Mrgpr-clusterΔ^{-/-} mice responded normally to noxious acute chemical stimuli. The writhing responses to intraperitoneal injection of acetic acid (0.6%, 15 ml/kg) were indistinguishable between WT and Mrgpr-clusterΔ^{-/-} mice (n=12). FIG. 2F is a histogram showing that Mrgpr-clusterΔ^{-/-} mice showed increased pain behavior in the second inflammatory phase of the formalin test (2%, 10 μl), though they responded normally in the first acute phase (n=10). FIG. 2G shows that Mrgpr-clusterΔ^{-/-} mice had higher numbers of c-fos⁺ cells in lumbar L4-L6 spinal cord ipsilateral to formalin injection as compared with WT. Positive staining was quantitated from three animals of each genotype. FIG. 2H is a graph showing that Mrgpr-clusterΔ^{-/-} mice displayed significantly stronger mechanical allodynia 24 hours after intraplantar injection of complete Freund's adjuvant (CFA, 6 μl, 50%) as compared with WT (n=13). FIG. 2I is a graph showing that CFA-induced thermal hyperalgesia was indistinguishable between WT and Mrgpr-clusterΔ^{-/-} mice within three days after injection. However, on the fourth day, WT mice recovered remarkably compared to Mrgpr-clusterΔ^{-/-} mice (n=13). FIG. 2J is a graph showing that Mrgpr-clusterΔ^{-/-} mice showed significantly stronger thermal hyperalgesia 24 hours after intraplantar injection of 1% carrageenen as compared with WT (10 μl, n=14). The data are presented as mean±SEM. *, p<0.05; ***, P<0.05; two-tailed unpaired t-test.

FIGS. 3A-D show that the wind-up responses of WDR neurons were enhanced in Mrgpr-clusterΔ^{-/-} mice. FIG. 3A are example of an analog recording of a WDR neuron displaying A-fiber-(0-40 msec) and C-fiber-mediated responses (40-2S0 msec) to an intra-cutaneous electrical stimulus (3.0 mA, 2.0 msec). There were no significant differences between WT (n=23) and Mrgpr-clusterΔ^{-/-} (n=30) mice in stimulus intensity-response functions of A-fiber- or C-fiber-mediated responses to the graded intra-cutaneous electrical stimuli. FIG. 3B are histograms showing representative responses of a WDR neuron in Mrgpr-clusterΔ^{-/-} displaying progressive increase in response (wind-up) to a train of electrical stimuli applied at a frequency of 0.2 Hz. In contrast, a WT WDR neuron did not show the wind-up response to 0.2 Hz stimulation. Bin size is 50 msec. FIG. 3C are graphs showing C-component responses of WDR neurons in WT and Mrgpr-clusterΔ^{-/-} mice to repetitive electrical stimulation applied at 0.2, 0.5 and 1.0 Hz. FIG. 3D is a graph showing the average of C-component responses to the last ten stimuli (7^{th}-16^{th}). Stimulation applied at a frequency of 0.2 Hz induced a significant level of wind-up in Mrgpr-clusterΔ^{-/-} but not WT mice compared to the baseline input (dashed line). Notably, the averaged C-component responses to the last ten stimuli of both 0.2 Hz and 1.0 Hz stimulation were significantly higher in Mrgpr-clusterΔ^{-/-} mice than that in WT mice. Wind-up data are normalized to the first response of each trial. *, p<0.05 and **, p<0.01, wind-up value compared with the input value; #, p<0.05, wind-up value compared with that of WT mice. Data are presented as mean±SEM.

FIGS. 4A-B illustrate the effect of RF-amide related peptides (agonists for Mrgpr receptors) on regulation of neuronal excitability. FIG. 4A provides histograms showing the percentages of P0 WT and Mrgpr-clusterΔ^{-/-} (KO) DRG neurons that responded to RF-amide related peptides BAM 8-22, NPFF, FMRFamide, and γ2-MSH 7-12 by increasing their intracellular calcium levels. The data was quantitated from 3-4 animals of each genotype. FIG. 4B provides graphs showing that FMRFamide increased firing rates in an Mrgpr-dependent manner in small-diameter primary sensory neurons. Action potentials were elicited with prolonged (500 ms) injections of a depolarizing current (200 pA). In WT mice, the average numbers of action potentials before and after the treatment were 3.6±1.6 and 7.7±1.9, respectively (n=10). However, Mrgpr-deficient neurons did not show an increase in firing rate.

FIGS. 5A-C are histograms showing that MrgprA1 does not play a major role in mediating the response of DRG neurons to RF-amide related peptides. FIG. 5A shows that the percentage of DRG neurons responding to RF-amide related peptides BAM 8-22, γ2-MSH 7-12, NPFF and FMRFamide did not differ significantly between WT and MrgprA1^{GFP/GFP} mice, in which the entire coding sequence of MrgprA1 was replaced with an in-frame fusion of GFP. FIG. 5B shows the percentages of peptide-responsive neurons found in the MrgprA1-GFP-expressing population. FIG. 5C shows that the percentages of MrgprA1^{GFP/GFP} neurons that responded to RF-amide related peptides. Data are presented as mean±SEM (n=3-4).

FIGS. 6A-D are histograms showing that intrathecal injection of BAM 8-22 inhibits persistent inflammatory pain and neuropathic pain in wild-type (WT) mice, but not Mrgpr-clusterΔ^{-/-} mice. FIG. 6A shows that intrathecal (i.th.) injection of BAM 8-22 (1 mM, 5 μl) significantly alleviated thermal hyperalgesia in the ipsilateral hind paw 24 h after intra-plantar injection of complete Freund's Adjuvant (CFA, 6μ, 50%) in WT (n=12), but not in Mrgpr-clusterΔ^{-/-} mice (n=10). BAM 8-22 did not affect paw withdrawal latency (PWL) of the contralateral hind paw in either group. FIG. 6B shows that the same dose of BAM 8-22 did not significantly change the tail flick latency in the tail immersion test (50° C.) in WT (n=10) or Mrgpr-clusterΔ^{-/-} mice (n=10). In addition, the tail flick latencies were not significantly different between the two groups at pre- and post-drug conditions. PWL of the contralateral hind paw to radiant heat (Hargreaves test) in the CFA experiment was similar before and after intrathecal BAM 8-22 injection in both groups. FIG. 6C shows that BAM 8-22 (0.5 mM, 5 μl, i.th.) attenuated mechanical pain hypersensitivity induced by chronic constriction injury of the sciatic nerve in WT mice but not in Mrgpr-clusterΔ^{-/-} mice. The paw withdrawal frequency (PWF) of the ipsilateral hind paw to low-force (0.07 g) and high-force (0.45 g) punctuate stimulation was significantly increased from the pre-injury levels in both Mrgpr-clusterΔ^{-/-} and WT mice 14-18 days post-injury. BAM 8-22 significantly reduced the PWF of the ipsilateral hind paw in response to low- and high-force stimuli in WT mice (n=7), but not in Mrgpr-clusterΔ^{-/-} mice (n=8), after 30 min. FIG. 6D shows that BAM 8-22 did not significantly reduce the PWF of the contralateral hind paw in either group. Data are expressed as mean±SEM. *P<0.05, **P<0.01 vs. pre-injury value; ##P<0.01 vs. pre-drug value.

FIGS. 7A-D are graphs showing that BAM 8-22 inhibits windup in wild-type (WT) mice. FIG. 7A provides graphs in which the C-components of WDR neuronal response to 0.5 Hz stimulation were plotted as a function of stimulus number before and after BAM 8-22 administration. FIG. 7B shows a histogram in which the averaged C-component responses for the last 10 stimuli during 0.5 Hz stimulation in WT mice were normalized by the respective response evoked by the first stimulation of each trial (input value). The relative windup in WT mice was significantly decreased by BAM 8-22, compared to the pre-drug level. Because of a significant increase of input in Mrgpr-clusterΔ^{-/-} mice after BAM 8-22 treatment, windup data were not normalized. FIG. 7C provides histograms showing an example of the inhibitory effect of BAM 8-22 on the windup of a WDR neuron in WT mice at 0.5 Hz stimulation. The windup response was substantially attenuated 10-30 min after BAM 8-22 application and was partially recovered 10-30 min after saline wash-out. Bin size is 50 msec. FIG. 7D provides graphs showing that BAM 8-22 (0.1 mM, 30 μl) significantly increased the C-component response to graded electrical stimulation at intensities of 2.0-5.0 mA in Mrgpr-clusterΔ^{-/-} mice (n=17), but not in WT mice (n=25), at 10-30 min after spinal topical application. Data are expressed as mean±SEM. *P<0.05 and **P<0.01 vs. the pre-drug condition; # P<0.05 vs. the input value.

FIG. 8A-B are graphs showing the effects of BAM 8-22 on C-component response of WDR neurons to electrical stimulation in WT and Mrgpr-clusterΔ^{-/-} mice. FIG. 8A provides graphs showing that spinal application of BAM 8-22 (0.1 mM, 30-50 μl) significantly increased acute C-component responses of WDR neurons to graded electrical stimulation at intensities of 2.0-5.0 mA in Mrgpr-clusterΔ^{-/-} mice (n=17) but not in WT mice (n=25). FIG. 8B provides graphs showing that spinal application of BAM 8-22 (0.1 mM, 30-50 μl) inhibited C-component responses of WDR neurons to 0.5 and 1.0 Hz stimulation in WT mice. In contrast, BAM 8-22 increased the input value and C-component responses to 0.5 and 1.0 Hz stimulation as compared with pre-drug responses in Mrgpr-clusterΔ^{-/-} mice. *, p<0.05, compared with pre-drug responses. Data are presented as mean±SEM.

### DETAILED DESCRIPTION

### Mas-Related G-Protein-Coupled Receptors (Mrgprs)

Mrgprs (also called sensory neuron specific receptors (SNSRs)) are a large family of orphan G-protein-coupled receptors (GPCRs). The Mrgpr gene family contains more than 50 members in the mouse genome, which can be grouped into several subfamilies: MrgprA1-22, MrgprB1-13, MrgprC1-14, and MrgprD-G (Dong et al., Cell 106:619-632, 2001; Zylka et al., Proc. Natl. Acad. Sci. USA 100:10043-10048, 2003). The Mrgpr family is smaller in other species such as rat and human, suggesting an atypical expansion of Mrgpr genes in mice (Dong et al., 2001; Zylka et al., 2003). The expression of Mrgprs, including MrgprAs, MrgprB4, MrgprB5, MrgprC11 and MrgprO, is restricted to subsets of small-diameter sensory neurons in dorsal root ganglia (DRG) and trigeminal ganglia, and has not been detected in the central nervous system (CNS) or in the rest of the body. The expression of Mrgprs in humans is also highly tissue- and/or cell-specific. For example, human MrgprX1, a homolog of mouse MrgprC11, is specifically expressed in a subset of small dorsal root and trigeminal sensory neurons. Human MrgprD was only found to be expressed in human dorsal root gangli neurons, but no in other human tissues tested (such as brain, heart, skeletal muscle, thymus, colon, spleen, kidney, liver, small intestine, placenta, lung, and peripheral blood leukocytes). The highly restricted expression of these receptors indicates that Mrgprs are involved in pain regulation.

As disclosed herein, members of the Mrgpr family, in particular MrgprX1 (a human homolog of mouse MrgprC11), are involved in an endogenous inhibitory mechanism for regulating persistent pain in mammals. In some embodiments, MrgprX1 is used to identify MrgprX1 agonists that reduce or inhibit persistent pain and, preferably, do not change perception of acute pain. In other embodiments, activation of Mrgpr receptors, such as MrgprX1, inhibits persistent pain. In some embodiments, deactivation or deletion of Mrgpr receptors, such as MrgprX1, enhances persistent pain. Agonists of Mrgpr receptors, such as an agonist of MrgprX1 (“MrgprX1 agonist”), can activate Mrgpr receptors in nociceptive neurons, and thus be used to treat a subject suffering from persistent pain or prevent persistent pain in a subject susceptible to persistent pain. Moreover, in some embodiments, prevention, inhibition or alleviation of persistent pain is achieved by using an agonist of Mrgpr receptor, such as an MrgprX1 agonist, that has no significant effect on acute pain.

### DEFINITIONS

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the terms “protein” and “polypeptide” are used interchangeably and refer to a polymer of amino acids. A polypeptide can be of various lengths. Thus, peptides, oligopeptides and proteins are included within the definition of polypeptide. A polypeptide can be with or without N-terminal methionine residues. A polypeptide may include post-translational modifications, for example, glycosylation, acetylation, phosphorylation and the like. Examples of “polypeptide” include, but are not limited to, polypeptides containing one or more analogs of an amino acid (including, for example, unnatural amino acids, non-coded amino acids, etc.), polypeptides with substituted linkages, fusion proteins, as well as polypeptides with other modifications known in the art, both naturally occurring and non-naturally occurring. The term “protein” or “polypeptide” also refer to naturally-occurring allelic variants and proteins that have a slightly different amino acid sequence than those specifically recited above. Allelic variants, though possessing a slightly different amino acid sequence than those recited above, will still have the same or similar biological functions associated with the protein.

Identity or homology with respect to amino acid sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

Proteins can be aligned, for example, using CLUSTALW (Thompson et al. Nucleic Acids Res 22:4673-80 (1994)) and homology or identity at the nucleotide or amino acid sequence level may be determined, for example, by BLAST (Basic Local Alignment Search Tool) analysis using the algorithm employed by the programs blastp, blastn, blastx, tblastn and tblastx (Karlin, et al. Proc. Natl. Acad. Sci. USA, 1990, 87:2264-2268 and Altschul, S. F. J. Mol. Evol., 1993, 36:290-300, both of which are herein incorporated by reference in its entirety) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is herein incorporated by reference in its entirety. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA, 1992, 89:10915-10919, which is herein incorporated by reference in its entirety). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

As used herein, the term “variant” refers to a biologically active polypeptide having an ammo acid sequence which differs from the sequence of a native sequence polypeptide disclosed herein, by virtue of an insertion, deletion, modification and/or substitution of one or more amino acid residues within the native sequence. Variants include peptide fragments of at least 5 amino acids, preferably at least 10 amino acids, more preferably at least 15 amino acids, even more preferably at least 20 amino acids that retain a biological activity of the corresponding native sequence polypeptide. Variants also include polypeptides wherein one or more amino acid residues are added at the N- or C-terminus of, or within, a native sequence. Further, variants also include polypeptides where a number of amino acid residues are deleted and optionally substituted by one or more different amino acid residues.

As used herein, the term “conservative variant” refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

As used herein, unless indicated otherwise, the terms “Mrgpr” and “Mrg” are used interchangeably and refer to any one or more of the mammalian mas-related gene (Mrg) receptors (i.e. MrgprA1-8, MrgprB, MrgprC, MrgprD, MrgprE, MrgprX1-4 and any other members of the mas-related gene (Mrg) family now known or identified in the future), including native mammalian sequences, such as murine or human Mrg receptors, Mrg receptor variants; Mrg receptor extracellular domain; and chimeric Mrg receptors.

As used herein, the terms “MrgprX1” and “MrgX1” are used interchangeably and refer to an amino acid sequence having at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, or about 100% sequence identity to a polypeptide described by NCBI Reference Sequence No. NP_{—}671732.3 (SEQ ID NO:1) or a fragment thereof that has MrgprX1 biological activity.

As used herein, “MrgprX1 nucleic acid molecule” refers to a polynucleotide sequence encoding an MrgprX1 polypeptide.

As used herein, the terms “polynucleotide” and “nucleic acid” are used interchangeably and refer to polymeric forms of nucleotides of any length. Thus, oligonucleotides are included within the definition of polynucleotide. “Nucleic acid” can be RNA or DNA that encodes a protein or peptide as defined above, is complementary to a nucleic acid sequence encoding such peptides, hybridizes to such a nucleic acid and remains stably bound to it under appropriate stringency conditions, exhibits at least about 50%, 60%, 70%, 75%, 85%, 90% or 95% nucleotide sequence identity across the open reading frame, or encodes a polypeptide sharing at least about 50%, 60%, 70% or 75% sequence identity, preferably at least about 80%, and more preferably at least about 85%, and even more preferably at least about 90 or 95% or more identity with the peptide sequences. Specifically contemplated are genomic DNA, eDNA, mRNA and antisense molecules, as well as nucleic acids based on alternative backbones or including alternative bases whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

As used herein, the terms nucleic acid, polynucleotide and nucleotide are interchangeable and refer to any nucleic acid, whether composed of phosphodiester linkages or modified linkages such as phosphotriester, phosphoramidate, siloxane, carbonate, carboxymethyl ester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyl-uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6:-diaminopurine.

Furthermore, a polynucleotide may comprise at least one modified sugar moiety selected from the group including but not limited to arabinose, 2-fluoroarabinose, xylulose, and hexose.

As used herein, a nucleic acid molecule is said to be “isolated” when the nucleic acid molecule is substantially separated from contaminant nucleic acid molecules encoding other polypeptides.

Highly related gene homologs are polynucleotides encoding proteins that have at least about 60% amino acid sequence identity with the amino acid sequence of a naturally occurring native sequence polynucleotide disclosed herein, preferably at least about 65%, 70%, 75%, 80%, with increasing preference of at least about 85% to at least about 99% amino acid sequence identity, in 1% increments.

As used herein, the term “antibody” is used herein in the broadest sense and specifically covers human, non-human (e.g., murine) and humanized antibodies, including, but not limited to, full-length monoclonal antibodies, polyclonal antibodies, multi-specific antibodies, and antibody fragments, including intrabodies, so long as they exhibit a desired biological activity. In general, an antibody exhibits binding specificity to a specific antigen.

As used herein, the term “subject” is a vertebrate, preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the mammal herein is human.

As used herein, the term “agonist” is used in the broadest sense and refers to any molecule or compound that fully or partially activates, stimulates, enhances, or promotes one or more of the biological properties of a polypeptide disclosed herein. Agonists may include, but are not limited to, small organic and inorganic molecules, nucleic acids, peptides, peptide mimetics and antibodies.

As used herein, the term “antagonist” is used in the broadest sense and refers to any molecule or compound that blocks, inhibits or neutralizes, either partially or fully, a biological activity mediated by a receptor of the present invention by preventing the binding of an agonist. Antagonists may include, but are not limited to, small organic and inorganic molecules, nucleic acids, peptides, peptide mimetics and neutralizing antibodies.

As used herein, the term “biological property” or “biological activity” refers to a biological function caused by a protein, such as an Mrgpr (including, but not limited to, MrgprC11 and MrgprX1), an agonist of an Mrgpr (including, but not limited to MrgprC11 agonists and MrgprX1 agonists), or other compound disclosed herein. Biological properties of Mrgprs include, but are not limited to, G-protein coupled receptor signal transduction activity, regulating the function or development of noceptive neurons, functioning as itch receptors, modulating opioid signaling, and regulating calcium-signaling pathway. With regard to agonists of Mrgprs, biological activity refers, in part, to the ability to fully or partially activate, stimulate, enhance, or promote the biological properties of Mrgprs. For example, an MrgprX1 agonist can have the ability to stimulate, enhance, or promote the activation of MrgprX1. Other preferred biologic activities of agonists of Mrgprs include, but are not limited to, treatment, alleviation, prevention or stopping persistent pain.

As used herein, the term “treatment” refers to a clinical intervention made in response to a disease, disorder or physiological condition manifested by a patient, particular persistent pain. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may alleviate pain, including pain resulting from an existing condition or disorder, or to prevent pain in situations where pain is likely to be experienced. As another example, in some embodiments, treatment may alleviate, prevent, slow, or stop persistent pain, including persistent pain resulting from or in association with other condition(s) or disorder(s), including, but not limited to, inflammation and nerve injury.

As used herein, the term “effective amount” or “effective dose” refers to an amount sufficient to effect beneficial or desirable clinical results. An effective amount of an agonist is an amount that is effective to treat a disease, disorder or unwanted physiological condition. In the case of persistent pain, the effective amount of an agonist of one or more Mrgprs, such as an MrgprX1 agonist, is sufficient to treat, prevent, alleviate or stop the symptom of persistent pain. The effective dose can be a single does, or can comprise multiple doses given over a period of time. In some embodiments, the amount used can be sufficient to activate one or more Mrgprs in the cell, tissue and/or the organism. In some embodiments, the amount used can be sufficient to activate MrgprX1 in the cell, tissue and/or the organism.

“Pharmaceutically acceptable” carriers, excipients, or stabilizers are ones which are nontoxic to the cell or mammal being exposed thereto at the dosages and concentrations employed. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™.

Pain is a sensation and a perception that is comprised of a complex series of mechanisms. Pain can be experienced both acutely and chronically. Acute pain is the instantaneous onset of a painful sensation in response to a noxious stimulus. It is considered to be adaptive because it can prevent an organism from damaging itself in some instances. Unlike acute pain (e.g., the transient protective physiology pain), persistent pain (also called chronic pain) usually has a delayed onset but can last for hours to days, or even months or years. Persistent pain may involve an amalgamation of physical, social, and psychologic factors. Persistent pain can be associated with conditions such as arthritis, nerve injury, AIDS and diabetes. Persistent pain occurs in a variety of forms including, but not limited to spontaneous pain (painful sensation without an external stimulus), allodynia (painful sensation in response to a normally innocuous stimulus) and hyperalgesia (strong painful sensation to a mildly painful stimulus). Persistent pain can be caused by many different factors. For example, persistent pain can be caused by conditions that accompany the aging process (for example conditions that may affect bones and joints in ways that cause persistent pain). As another example, persistent pain may also be caused by conditions, such as rheumatoid arthritis, osteoarthritis, cancer, multiple sclerosis, stomach ulcers, fungal infections, bacterial infections, viral infections (such as AIDS, hepatitis), and gallbladder disease. In some embodiments, persistent pain can be caused by inflammation or nerve injury (for example, damage to or malfunction of the nervous system). In some embodiments, persistent pain can be inflammatory pain or neuropathic pain (for example, peripheral neuropathic pain and central neuropathic pain). In some embodiments, persistent pain is mediated by hyper-excitable pain-processing neurons in peripheral and central nervous system (e.g., peripheral sensitization, central sensitization).

### Agonists of Mrgprs

As discussed above, the term “agonist” is used herein in a broad sense and includes any molecule that partially or fully activates a biologically activity mediated by one or more Mrgprs, such as MrgprX1. The term “agonist” also includes any molecule that mimics a biological activity mediated by an Mrgpr, such as MrgprX1, and molecules that specifically change, preferably increase, the function or expression of the Mrgpr, or the efficiency of signaling through the Mrgpr.

In some embodiments, agonists of Mrgprs, particularly MrgprX1 agonists, can be used to screen for compounds that reduce or inhibit persistent pain. Preferably such agonists are also screened to identify those agonists that do not significantly change the perception of acute pain. Such agonists of Mrgprs can be used to stimulate, enhance, or promote the biological properties of the Mrgprs. In some embodiments, agonists of Mrgprs can be used to directly activate Mrgpr receptors. In some embodiments, a first MrgprX1 agonist can be used to enhance the activation of MrgprX1, or other Mrgpr, by a second agonist. In some embodiments, agonists of Mrgprs, such as MrgprX1 agonists, can be used to positively allosterically modulate MrgprX1 or another Mrgpr. In other words, the agonist of Mrgprs can interact with one or more Mrgpr to increase the Mrgpr activation triggered by another Mrgpr agonist or Mrgpr binding partner. In some embodiments, an agonist of Mrgprs can bind to an Mrgpr's allosteric site and enhance the ability of an Mrgpr agonist to activate one or more biological properties of the Mrgpr. In some embodiments, an MrgprX1 agonist that is identified as able to inhibit persistent pain can be used to treat, prevent, or ameliorate persistent pain. In some embodiments, the treatment of persistent pain has no significant effect on acute pain.

The biological activity mediated by Mrgprs (including MrgprC11 and MrgprX1) may be activated by an agonist in any of a variety of ways. In some embodiments, an Mrgpr agonist can act directly on an Mrgpr (for example, by binding to the Mrgpr) and trigger the receptor activity of the Mrgpr. For example, an MrgprX1 agonist can act directly on MrgprX1. Non-limiting examples of such Mrgpr agonists include RF/Y-G or RF/Y-amide, such as the molluscan peptide FMRFamide, and the mammalian peptides neuropeptide FF (NPFF), neuropeptide AF (NPAF), 2-melanocyte-stimulating hormone (y2-MSH) and bovine adrenal medulla peptide (BAM). In some embodiments, an Mrgpr agonist can enhance the ability of an Mrgpr to interact with a ligand of the Mrgpr receptor. In some embodiments, a first Mrgpr agonist can enhance the activation of the Mrgpr by a second Mrgpr agonist.

In some embodiments, an Mrgpr agonist can be a constitutively active mutant Mrgpr, for example a constitutively active mutant MrgprX1. In some embodiments, an Mrgpr agonist can modulate the level of Mrgpr gene expression, preferably increasing the level of transcription of an Mrgpr gene. In some embodiments, an Mrgpr agonist can modulate the levels of an Mrgpr protein, such as MrgprX1 protein, in cells, tissues or the body of a subject by, for example, increasing the translation of the Mrgpr mRNA, or decreasing the degradation of Mrgpr mRNA or Mrgpr protein.

Various molecules are known to activate Mrgprs. For example, Mrgprs can be activated by peptides terminating in RF/Y-G or RF/Y-amide, such as the molluscan peptide FMRFamide, and the mammalian peptides neuropeptide FF (NPFF), neuropeptide AF (NPAF), γ2-melanocyte-stimulating hormone (y2-MSH) and bovine adrenal medulla peptide (BAM). These peptides can activate heterologously expressed mouse MrgprA1, MrgprA4 and MrgprC11, and human MrgprX1 receptors with different sensitivities (Dong et al, 2001; Han et al, Proc. Natl. Acad. Sci. USA 99:14740-14745, 2002; Lembo et al., Nat. Neurosci. 5:201-209, 2002). In some embodiments, such molecules (e.g., BAM 22, and BAM 8-22) activate MrgprX1, when they are delivered into the spinal cord of a subject. In some embodiments, such activation of MrgprX1 prevents, inhibits or alleviates persistent pain, but has no significant effect on acute pain.

An endogenous agonist of MrgprC11 is a 22-amino acid peptide called bovine adrenal medulla peptide (BAM22). BAM22 belongs to the family of endogenous opioid peptides and is derived from the proenkephalin A gene. The N-terminus of BAM22 (BAM 1-7) binds and activates opioid receptors whereas the C-terminus of the peptide (BAM 8-22) specifically activates mouse MrgprC11, rat MrgprC, and human MrgprX1, but not opioid receptors (Han et al, 2002; Lembo et al., 2002).

In some embodiments, an MrgprX1 agonist interacts with MrgprX1 directly and triggers the activation of MrgprX1. In some embodiments, an MrgprX1 agonist may enhance the interaction of MrgprX1 with a binding partner or ligand (for example, BAM22, BAM 8-22 and chloroquine); or enhance the MrgprX1 gene expression; or increase the number of MrgprX1 receptors on the cell surface; or modulate the level of MrgprX1 protein in the cell, tissue or body of a subject. In some embodiments, the MrgpX1 agonist may interact with a compound that is in an MrgprX1 dependent pathway, for example, upstream or downstream from MrgprX1. In some other embodiments, an MrgprX1 agonist may bind to MrgprX1 directly to trigger the activation of MrgprX1. In still other embodiments, an MrgprX1 agonist may bind to MrgprX1 to enhance the activation of MrgprX1 triggered by a second MrgprX1 agonist.

In some embodiments, the MrgprX1 agonist can be a positive allosteric modulator of a second MrgprX1 agonist. In some embodiments, an MrgprX1 agonist can enhance the gene expression or the level of a second MrgprX1 agonist in the body of a subject. For example, the MrgprX1 agonist may increase the level of BAM22 in the cell, tissue or body of a subject by enhancing the gene expression of the proenkephalin A gene, decreasing the degradation or turnover of the proenkephalin A mRNA or protein, or increasing the cleavage of proenkephalin A to produce BAM 22.

The types of MrgprX1 agonists are not limited in any way. Non-limiting examples of MrgprX1 agonists include small molecules (including both organic and inorganic molecules), peptides, peptide mimetics, proteins, nucleic acids, and antibodies. In some embodiments, the MrgprX1 agonist is a small molecule that binds to MrgprX1. For example, the MrgprX1 agonist can be a small molecule MrgprX1 agonist disclosed in Wroblowski et al. (J. Med. Chem., 2009, 52:818-825, which is hereby incorporated by reference in its entirety. In some embodiments, the MrgprX1 agonist can be a compound of formula I, or a pharmaceutically acceptable salt thereof:

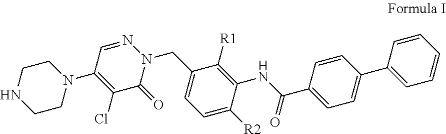

wherein R1 can be H or CH₃, and R2 can be H or CH₃.

In some embodiments, the MrgprX1 agonist can be a compound of formula II, or a pharmaceutically acceptable salt thereof:

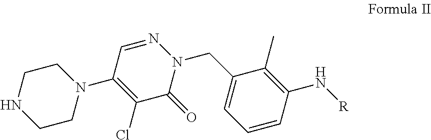

wherein R can be

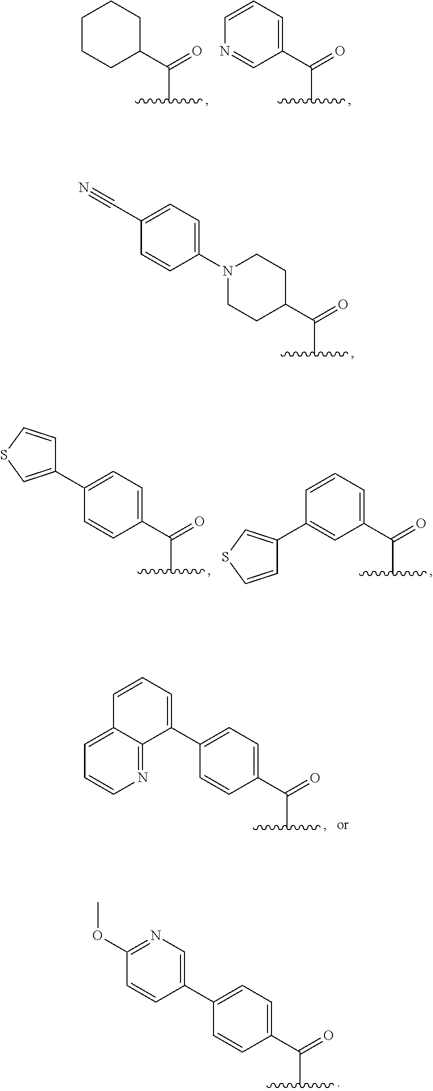

In some embodiments, the MrgprX1 agonist can be a compound of formula III, or a pharmaceutically acceptable salt thereof:

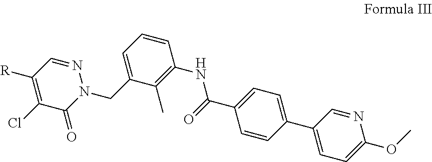

wherein R can be

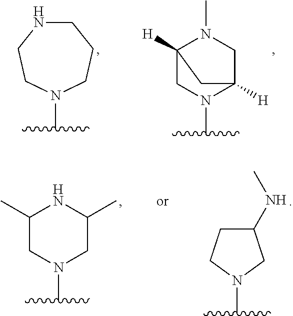

In some embodiments, the MrgprX1 agonist can be a compound of formula IV, or a pharmaceutically acceptable salt thereof:

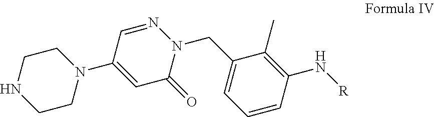

wherein R can be

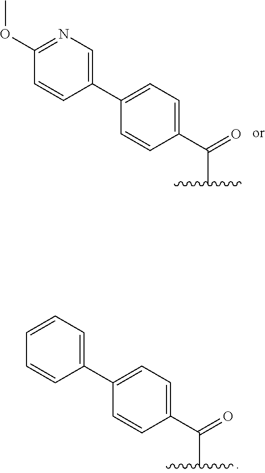

In some embodiments, the MrgprX1 agonist can be 1-(2-(2-Methyl-3-(4-phenylbenzoylamido)-benzyl)-4-chloro-3-ox-opyridazin-5-yl)-piperazine, N-[3-(5-Chloro-6-oxo-4-piperazin-1-yl-6H-pyridazin-1-ylmethyl)-2-methyl-phenyl]-4-(6-methoxy-pyridin-3-yl)-benzamide, N-[3-(6-oxo-4-Piperazin-1-yl-6H-pyridazin-1-ylmethyl)-2-methyl-phenyl]-4-(6-methoxy-pyridin-3-yl)-benzamide, or Biphenyl-4-carboxylic acid [3-(6-oxo-4-piperazin-1-yl-6-pyridazin-1-ylmethyl)-2-methyl-phenyl]-amide.

As other examples, the MrgprX1 agonist can be a small molecule MrgprX1 agonist disclosed in Malik et al. (Bioorganic & Medicinal Chemistry Letters 2009, 19:1729-1732, the entire content of which is hereby incorporated by reference). In some embodiments, the MrgprX1 agonist can be a compound of formula V, or a pharmaceutically acceptable salt thereof:

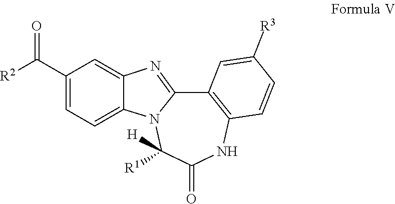

wherein R¹ can be

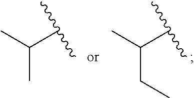

### R² can be

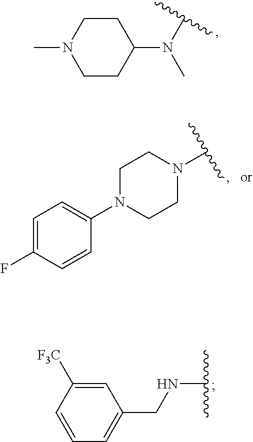

### and R³ can be

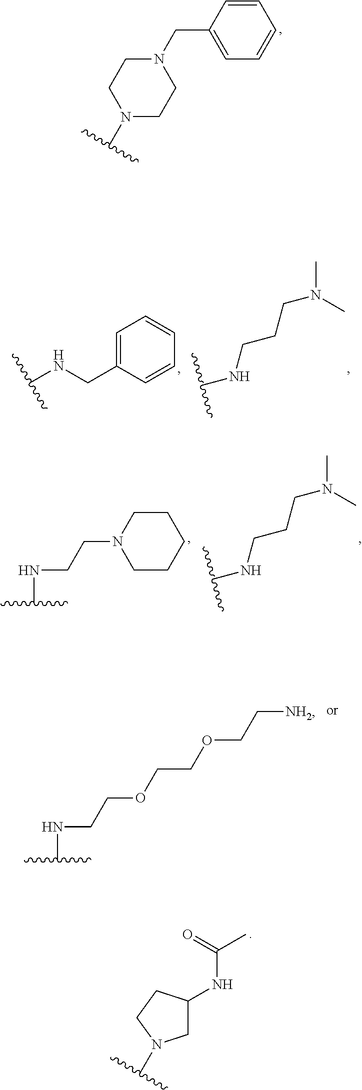

As other examples, the MrgprX1 agonist can be a benzoimidazole compound having the ability of modulating the activity of MrgprX1. Some examples of such benzoimidazole compounds are disclosed in U.S. Patent Publication US 2008/0249081, which is incorporated by reference herein in its entirety.

In some embodiments, the MrgprX1 agonist can be a peptide. For example, the MrgprX1 agonist can be BAM22 or BAM 8-22. In addition, a peptide called P60 and described in Shemesh et al., the Journal of Biological Chemistry, 2008, 283(50): 34643-34649 (Swiss Prot ID: CE029_HUMAN; GIGCVWHWKHRVATRFTLPRFLQ; SEQ ID NO:2) has been shown to be able to activate MrgprX1, but does not activate any of the known opioid receptors, such as D1, M1, L1, and K1 opioid receptors.

### Screening Assays for Agonists of Mrgprs

In some embodiments, known Mrgpr agonists, such as known MrgX1 agonists are used in the disclosed methods. However, in other embodiments agonists are identified by screening compounds for their ability to act as Mrgpr agonists, particularly MrgprX1 agonists. Screening assays are well known in the art and can readily be adapted to identify agonists of Mrgprs, such as MrgprX1. As discussed above, agonists of Mrgprs may include compounds that interact with (e.g., bind to) an Mrgpr; compounds that enhance the interaction of an Mrgpr with its binding partner, cognate or ligand (e.g., a positive allosteric modulator of an Mrgpr ligand or an Mrgpr agonist); and compounds that modulate, preferably increase, the level of Mrgpr in the cell, tissue or body of a subject, such as compounds that modulate Mrgpr gene expression. Assays may additionally be utilized to identify compounds that bind to Mrgpr gene regulatory sequences (e.g., promoter sequences) and, consequently, may modulate Mrgpr gene expression.

The compounds which may be screened include, but are not limited to small molecules (including both organic and inorganic molecules), peptides, proteins, antibodies and fragments thereof, and other organic compounds (e.g., peptidomimetics). The compounds can include, but are not limited to, soluble peptides, including members of random peptide libraries (see e.g., Lam, K. S. et al., 1991, Nature 354:82-84; Houghten, R. et al., 1991, Nature 354:84-86), and combinatorial chemistry-derived molecular libraries made of D- and/or L-configuration amino acids, phosphopeptides (including, but not limited to members of random or partially degenerate, directed phosphopeptide libraries; see e.g., Songyang, Z. et al., 1993, Cell 72:767-778), antibodies (including, but not limited to, polyclonal, monoclonal, humanized, anti-idiotypic, chimeric or single chain antibodies, and FAb, F(abN)₂ and FAb expression library fragments, and epitope-binding fragments thereof), and small organic or inorganic molecules, including libraries thereof. Other compounds that can be screened in accordance with the present application include, but are not limited to, small organic molecules, for example, those that are able to cross the blood-brain barrier.

Many methods are available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of known compounds, including natural products or synthetic chemicals, and biologically active materials, including proteins, can be screened for compounds which can activate Mrgprs, including MrgprX1.

Small molecules can also have the ability to activate Mrgprs (including MrgprX1) and thus may be screened for such activity. In some embodiments, small molecules can have a molecular weight of less than about 10 kD, about 8 kD, about 5 kD, and about 2 kD. Such small molecules may include naturally-occurring small molecules, synthetic organic or inorganic compounds, peptides and peptide mimetics. However, small molecules in the present application are not limited to these forms. Extensive libraries of small molecules are commercially available and a wide variety of assays are well known in the art to screen these molecules for the desired activity.

In some embodiments, agonists of Mrgprs (e.g., compounds that specifically bind and activate an Mrgpr polypeptide, such as an MrgprX1 agonist) are identified from large libraries of natural product or synthetic (or semisynthetic) extracts or chemical libraries or from polypeptide or nucleic acid libraries, according to methods known in the art. Those skilled in the field of drug discovery and development will understand that the precise source of test extracts or compounds is not critical to the screening procedure(s) disclosed herein. Agents used in screens may include those known as therapeutics for the treatment of persistent pain. Virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al, Proc. Natl. Acad. Set U.S.A. 90:6909, 1993; Erb et al, Proc. Natl. Acad. Sci. USA 91:11422, 1994; Zuckermann et al, J. Med. Chem. 37:2678, 1994; Cho et al, Science 261:1303, 1993; Carrell et al, Angew. Chem. Int. Ed. Engl. 33:2059, 1994; Carell et al, Angew. Chem. Int. Ed. Engl. 33:2061, 1994; and Gallop et al, J. Med. Chem. 37:1233, 1994. Furthermore, if desired, any library or compound is readily modified using standard chemical, physical, or biochemical methods.

In some embodiments, candidate agonist compounds can be identified by first identifying those that specifically bind to an Mrgpr polypeptide, particularly an MrgprX1 polypeptide and subsequently testing their effect on MrgprX1 biological activity (e.g., using Ca²⁺ influx). The interaction of a compound with an Mrgpr polypeptide can be readily assayed using any number of standard binding techniques and functional assays well known in the art.

In some embodiments, a candidate compound that binds to an MrgprX1 polypeptide may be identified using a chromatography-based technique. For example, an MrgprX1 polypeptide may be purified by standard techniques from cells engineered to express the polypeptide, or may be chemically synthesized, once purified the peptide is immobilized on a column. A solution of candidate compound is then passed through the column, and a compound that specifically binds the MrgprX1 polypeptide or a fragment thereof can be identified on the basis of its ability to bind to MrgprX1 polypeptide and to be immobilized on the column. To isolate the compound, the column can be washed to remove non-specifically bound molecules, and the agent of interest is then released from the column and collected. The compound isolated by this method (or any other appropriate method) may, if desired, be further purified (e.g., by high performance liquid chromatography). In addition, these candidate compounds may be tested for their ability to modulate MrgprX1 (e.g., as described herein).

A candidate compound that specifically binds to MrgprX1 can be tested for activity in an in vitro assay or in vivo assay for its ability to activate MrgprX1, and subsequently for its ability to treat or reduce persistent pain, as discussed below. For example, a candidate compound may be tested in vitro for interaction and binding with a Mrgpr polypeptide disclosed herein and then for its ability to modulate MrgprX1 activity. A variety of methods well known in the art can be used to measure the ability of a molecule to activate an Mrgpr receptor, such as MrgprX1. The ability to modulate MrgprX1 activity may be assayed in vitro by any standard assay for G-protein coupled receptor activity, such as Ca²⁺ influx assay, or by patch clamp or other assay for electrical activity. In some embodiments, the test compounds can be screened using HEK293 cells stably transfected with human MrgprX1 in an intracellular calcium mobilization assay with the fluorometric imaging plate reader (FLIPR, Molecular Devices) as described by Sulivan et al (J. Mol. Biol. 1993, 234:779-815).

In some embodiments, the level of MrgprX1 activation by a potential MrgprX1 agonist can be measured by methods described in Wroblowski et al. (J. Med. Chem., 2009, 52:818-825). As another example, an agonist of Mrgprs (such as an MrgprX1 agonist) can be identified using gene reporter assay and function receptor assay. In an example, a function receptor assay called Receptor Selection and Amplification Technology (R-SAT) can be used to detect the extent a test compound can activate Mrgpr and compare the activation of Mrgpr achieved by the test compound with that achieved by a known MrgprX1 agonist, such as BAM22). R-SAT is described in detail in U.S. Pat. Nos. 5,707,798; 5,912,132; and 5,955,281, each of which is incorporated by reference herein in its entirety. Examples of detecting the agonist activity of test compounds using NIH3T3 cells transfected with a human MrgprX1 reporter gene and examples of MrgprX1 receptor binding assay are described in U.S. Patent Application Publication No. 2008/0249081, which is incorporated by reference herein in its entirety.

One skilled in the art will appreciate that the effects of a candidate compound on a cell is typically compared to a corresponding control cell not contacted with the candidate compound. The screening methods include, but are not limited to, comparing Ca²⁺ influx in an MrgprX1-expressing cell contacted by a candidate agent with Ca²⁺ influx in an untreated control cell. In some embodiments, the expression or activity of MrgprX1 in a cell treated with a candidate compound is compared to untreated control samples to identify a candidate compound that increases the expression or activity of MrgprX1 in the contacted cell. Polypeptide or polynucleotide expression can be compared by procedures well known in the art, such as Western blotting, flow cytometry, immunocytochemistry, binding to magnetic and/or MrgprX1-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

After identification, agonists may be screened for their ability to treat persistent pain, as described below. In some embodiments, compounds are also screened to determine whether or not they change perception of acute pain.

Testing Candidate Compounds for their Ability to Treat Persistent Pain

Once identified or provided, an Mrgpr agonist, such as MrgprX1 agonist, may be further tested for its ability to prevent, delay, ameliorate, stabilize, or treat disease or disorder characterized by persistent pain. Compounds that are found to modulate persistent pain may be used to stop or reduce persistent pain in a patient suffering from persistent pain.

A candidate compound that has been found to activate an Mrgpr, particularly MrgprX1, can be administered to an animal in an animal model of persistent pain and tested for its ability to reduce persistent pain. In some embodiments, a candidate compound can be directly administered to the spinal cord of an animal in an animal model of persistent pain and the ability of the candidate compound to treat or reduce persistent pain can be tested, for example by determining spinal neuronal sensitization involved in persistent pain in the animal model

Various methods known in the art can be used to test a candidate compound, such as a known or suspected MrgprX1 agonist, for its ability to reduce persistent pain. In some embodiments, the candidate compound is tested in an animal model of persistent pain, for example, an animal suffering from persistent pain caused by inflammation or nerve injury. In some embodiments, the anti-hyperalgesic effect of a candidate compound can be determined at the level of central nociceptive processing. For example, the candidate compound can be delivered directly to the spinal cord of the animal and the effects of spinal application of the candidate compound on the wind-up responses of WDR neurons to repetitive noxious inputs can be determined for evaluating the anti-hyperalgesic effect of the candidate compound. Some exemplary methods are described in the Examples below.

In another non-limiting example, a chronic constriction injury (CCI) model of neuropathic pain in mice (for example, as described below in greater detail in the Examples below) can be used in testing candidate compounds for their ability to treat persistent pain. A candidate compound to be tested, for example BAM 8-22, can be injected intrathecally and delivered directly to spinal cord of the CCI mice. Then, behavioral studies, such as a formalin test, CFA-induced heat hyperalgesia (e.g., Hargreaves test), tail immersion test, and CCI-induced mechanical allodynia (e.g., von Frey test) can be performed to determine the anti-hyperalgesic effect of a candidate compound by comparing the results obtained from the CCI mice treated with candidate compounds with those from untreated control samples to identify a candidate compound that treats or reduces persistent pain.

In other embodiments, the spontaneous activity of wide dynamic range (WDR) neurons can be determined by electrophysiological recording to test anti-hyperalgesic effect of the candidate compound. In other embodiments candidate compounds can be administered prior to creation of the persistent pain state, in order to evaluate the ability of the compound to prevent the development of persistent pain.

In some embodiments, candidate compounds that reduce persistent pain are selected. In other embodiments, compounds that do not reduce persistent pain are eliminated from consideration as therapeutic agents for the treatment of persistent pain.

In some embodiments, Mrgpr agonists are tested for their ability to treat and/or prevent persistent pain in two or more animal models of persistent pain.

Compounds identified as a compound capable of preventing and/or reducing persistent pain may be used, for example, as therapeutics to treat or prevent the onset of a disease or disorder characterized by persistent pain.

Compounds that have been identified as Mrgpr agonists, particularly MrgprX1 agonists, may be further tested for their ability to modulate (increase or decrease) the perception of other types of pain. Such tests may be done concurrently with the tests for the ability to reduce persistent pain, or subsequent to the tests for the ability to reduce persistent pain. In some embodiments, compounds that have been identified as able to reduce persistent pain are tested for their ability to modulate acute pain, for example in animal models of acute pain. Tests for acute pain are well known in the art and include, for example, the tail-flick test, paw withdrawal test, hot plate test, and the writhing test. See, for example, Le Bars et al. Pharmacological Reviews 2001 53(4):597-652 for a review. Briefly, an animal is injected with the candidate compound and their response to the test of acute pain is compared to control animals. Compounds that are able to reduce persistent pain, but do not produce any appreciable or significant change in the perception of acute pain are selected for use as therapeutic compounds for the treatment of persistent pain.

### Compositions Comprising Agonists of Mrgprs

In some embodiments, a method of treatment of persistent pain comprises administration of an effective amount of a composition comprising one or more agonists of Mrgprs, such as one or more MrgprX1 agonists, that have been identified as reducing persistent pain. In some embodiments the agonists have also been identified as not significantly changing the perception of acute pain. In some embodiments a therapeutic amount of an MrgX1 agonist is administered to a patient identified as suffering from persistent pain. In some embodiments, the composition is administered indirectly to spinal cord. In some embodiments, the composition is administered directly to spinal cord. In some embodiments, the composition is administered directly to small diameter sensory neurons in DRG and trigeminal ganglia. In some embodiments, the composition is administered directly to the subsets of small diameter sensory neurons in DRG and trigeminal ganglia in which MrgprX1 is specifically expressed.

In some embodiments, the composition comprises at least one agonist of MrgprX1. In some embodiments, the MrgprX1 agonist can be a small molecule. For example, the MrgprX1 agonist can be a benzoimidazole compound capable of activating MrgprX1. Non-limiting examples of benzoimidazole compounds capable of activating MrgprX1 are described in U.S. Patent Publication No. 2008/0249081 (which is herein expressly incorporated by reference in its entirety). In some embodiments, the MrgprX1 agonist can be a peptide. For example, the MrgprX1 agonist can be BAM22, BAM 8-22, and peptide P60 comprising the amino acid sequence of SEQ ID NO:2.

In some embodiments, the composition comprises an MrgprX1 agonist that is positive allosteric modulator of a second MrgprX1 agonist. In some embodiments the positive allosteric modulator is administered in combination with the second MrgprX1 agonist.

In still other embodiments, the composition comprises an MrgprX1 agonist that is a nucleic acid. For example, the MrgprX1 agonist can be a nucleic acid that binds to the regulatory sequence of an Mrgpr gene and increases the transcription of the Mrgpr gene. As another example, the MrgprX1 agonist can be a nucleic acid that can decrease the degradation of MrgprX1 mRNA.

Therapeutic compositions can comprise any agonists of MrgprX1 identified by the methods described herein, and combinations thereof. In some embodiments, an agonist of MrgprX1 is included in an amount suitable for the treatment of persistent pain but that does not change the perception of acute pain significantly. In some embodiments, the agonist of Mrgprs is combined with other ingredients that are suitable for the treatment of persistent pain, such as inflammatory and neuropathic pain.

In pharmaceutical dosage forms, the agonists of Mrgprs can be used alone or in appropriate association, as well as in combination with other pharmaceutically active or inactive compounds. The agonists of Mrgprs can be formulated into pharmaceutical compositions containing a single agonist of Mrgprs or a combination of two or more agonists of Mrgprs. For example, a pharmaceutical composition can contain two or more different agonists of Mrgprs. In some embodiments, the pharmaceutical composition contains two or more different agonists of Mrgprs having the same mode of action. For example, a pharmaceutical composition can contain two agonists of Mrgprs where both agonists of Mrgprs are Mrgpr ligands and activate the Mrgpr directly. As another example, a pharmaceutical composition can contain two agonists of Mrgprs where one of the agonist of Mrgprs is a ligand of Mrgpr to active Mrgpr directly and the other agonist of Mrgprs is a positive allosteric modulator of Mrgpr that increases the activity of Mrgpr indirectly via activation of an allosteric site on Mrgpr. In other embodiments, the pharmaceutical composition can contain two or more agonists of Mrgprs having different methods of action. For example, one agonist of Mrgprs can be an Mrgpr ligand, while the other agonist of Mrgprs can be a compound that increases the gene expression of proenkephalin A and therefore increase the level of BAM 22.

The agonists of Mrgprs can be formulated into pharmaceutical compositions by combination with appropriate, pharmaceutically acceptable carriers or diluents (Remington, The Science and Practice of Pharmacy, 19.sup.th Edition, Alfonso, R., ed., Mack Publishing Co., Easton, Pa. (1995), and can be formulated into preparations in solid, semi-solid, liquid or gaseous forms, such as tablets, capsules, powders, granules, ointments, solutions, suppositories, injections, inhalants and aerosols depending on the particular circumstances.

The pharmaceutically acceptable excipients, such as vehicles, adjuvants, carriers or diluents, are readily available. Moreover, pharmaceutically acceptable auxiliary substances, such as pH adjusting and buffering agents, antioxidants, low molecular weight (less than about 10 residues) polypeptides, tonicity adjusting agents, stabilizers, wetting agents and the like, are readily available. “Carriers” when used herein refers to pharmaceutically acceptable carriers, excipients or stabilizers which are nontoxic to the cell or mammal being exposed to the carrier at the dosages and concentrations used.

An agonist of Mrgprs to be used for in vivo administration is preferably sterile. The sterility can be accomplished by any method known in the art, such as by filtration using sterile filtration membranes, prior to or following lyophilization and reconstitution. In some embodiments the agonists of Mrgprs are available commercially in sterile form.

The compositions containing one or more agonists of Mrgprs can be placed into a container with a sterile access port, for example, an intravenous solution bag or vial having a stopper pierceable by a hypodermic injection needle.

The agonists of Mrgprs can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agonists of Mrgprs can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing or dispersing agents. Alternatively, the active ingredient can be in powder form for constitution with a suitable vehicle, for example, sterile pyrogen-free water, before use. The compounds can also be formulated in rectal compositions such as suppositories or retention enemas, for example, containing conventional suppository bases such as cocoa butter or other glycerides.

In addition to the formulations described above, the agonists of Mrgprs can also be formulated as a depot preparation. Such long-acting formulations can be administered by implantation (for example, subcutaneously or intramuscularly) or by intramuscular injection. Thus, for example, the agonists of Mrgprs can be formulated with suitable polymeric or hydrophobic materials (for example as an emulsion in an acceptable oil) or ion exchange resins, or as sparingly soluble derivatives, for example, as a sparingly soluble salt.

For oral preparations, the agonists of Mrgprs can be combined with appropriate additives to make tablets, powders, granules or capsules. For example, the agonists of Mrgprs can be combined with conventional additives such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Liquid preparations for oral administration can take the form of, for example, solutions, syrups or suspensions, or they can be presented as a dry product for constitution with water or other suitable vehicle before use. Preparations for oral administration can be suitably formulated to give controlled release of the active compound.

The agonists of Mrgprs can also be aerosolized or otherwise prepared for administration by inhalation. For example a fluorocarbon formulation and a metered dose inhaler, or inhaled as a lyophilized and milled powder. For administration by inhalation, the agonists of Mrgprs can be utilized in aerosol formulation to be administered via inhalation. The agonists of Mrgprs can also be formulated into pressurized acceptable propellants such as dichlorodifluoromethane, propane, nitrogen and the like.

If an agonist of Mrgprs is coadministered with another agonist of Mrgprs, or with another agent having similar biological activity, the different active ingredients can be formulated together in an appropriate carrier vehicle to form a pharmaceutical composition. Alternatively, the agonists of Mrgprs can be formulated separately and administered simultaneously or in sequence.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In some embodiments, the agonist of Mrgprs is formulated for cellular use, and need not be formulated for administration to a subject. In some embodiments, the agonist of Mrgprs is formulated for direct application into the brain, e.g., direct injection or pump based delivery systems and methods. In some embodiments, the agonist of Mrgprs is formulated for or applied via intraventricular application.

### Methods of Treatment

In some embodiments, methods of treating (including preventing, (meaning reducing the risk of or time of onset of) an individual suffering from or at risk of persistent pain, such as inflammatory or neuropathic pain, are provided. The methods generally comprise administering to the individual one or more agonists of Mrgprs, such as one or more MrgprX1 agonists that have been identified as being able to reduce persistent pain. The agonists have also preferably been identified as not having a significant effect on acute pain. In some embodiments, a composition is administered that comprises one or more agonists of Mrgprs at a therapeutically effective dose. In some embodiments, the composition is administered directly into the spinal cord. In other embodiments, the composition is administered indirectly into the spinal cord. In some embodiments, the composition is administered to nociceptive neurons. In some embodiments, the composition is administered to small-diameter sensory neurons in DRG and/or trigeminal ganglia.

As discussed above, treatment can include an amelioration of the symptoms associated with the pathological condition afflicting the host, where amelioration is used in a broad sense to refer to at least a reduction in the magnitude of a parameter, e.g., symptom, associated with the pathological condition being treated, such as neuronal cell death. As such, treatment includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition. However, treatment can also be delay or slowing of disease progression, amelioration or palliation of the disease state, and remission (whether partial or total), whether detectable or undetectable.

A variety of individuals are treatable. Generally, such individuals are mammals, where the term is used broadly to describe organisms which are within the class mammalia, including the orders carnivore (for example, dogs and cats), rodentia (for example, mice, guinea pigs and rats), and primates (for example, humans, chimpanzees and monkeys). In preferred embodiments, the individuals are humans.

The agonists of Mrgprs can be administered using any convenient protocol capable of resulting in the desired therapeutic activity. A specific protocol can readily be determined by a skilled practitioner without undue experimentation based on the particular circumstances. Thus, the agonists of Mrgprs can be incorporated into a variety of formulations for therapeutic administration, as discussed above, depending on the protocol adapted by the supervising clinician. In some embodiments, the agonist of Mrgprs, such as MrgprX1 agonist, can be dissolved in saline solution and delivered directly or indirectly into the spinal cord.

Each dosage for human and animal subjects preferably contains a predetermined quantity of one or more agonists of Mrgprs calculated in an amount sufficient to produce the desired effect, in association with a pharmaceutically acceptable diluent, carrier or vehicle. Again, the actual dosage forms will depend on the particular compound employed, the effect to be achieved, and the pharmacodynamics associated with each compound in the host.

Administration of agonists of Mrgprs can be achieved in various ways, including intracranial, for example injection directly into the brain tissue or into the spinal cord, into the cerebrospinal fluid, oral, buccal, rectal, parenteral, intraperitoneal, intradermal, transdermal, intracheal, intracerebral, etc., administration. The agonists of Mrgprs can be administered alone or in combination with one or more additional therapeutic agents. Administration “in combination with” one or more further therapeutic agents includes both simultaneous (at the same time) and consecutive administration in any order.

Administration can be chronic or intermittent, as deemed appropriate by the supervising practitioner, particularly in view of any change in the disease state or any undesirable side effects. “Chronic” administration refers to administration of one or more agonists of Mrgprs in a continuous manner while “intermittent” administration refers to treatment that is not done without interruption.

Combinations of agonists of Mrgprs for simultaneous administration are used in some embodiments. For example, two or more different agonists of Mrgprs can be administered in combination.

In some embodiments, one or more agonists of Mrgprs are administered by intracspinal injection. The injection will typically be directly into the spinal cord or into the cerebrospinal fluid.

An effective amount of an agonist of Mrgprs to be employed therapeutically will depend, for example, upon the therapeutic objectives, the route of administration, the nature of the agonist of Mrgprs, and the condition of the patient. Accordingly, it can be useful for the therapist to titer the dosage and modify the route of administration as required to obtain the optimal therapeutic effect. A typical daily dosage can range from about 0.01 μg/kg to up to about 1 mg/kg or more, depending on the factors mentioned above. Preferably, a typical daily dosage ranges from about 1 μg/kg to about 100 μg/kg. Typically, the clinician will administer an agonist of Mrgprs until a dosage is reached that provides the best clinical outcome. The progress of this therapy is easily monitored by conventional assays. In some embodiments, a typical daily dosage of agonist of Mrgprs, for example MrgprX1 agonist, is from about 1 μM to about 10 mM. In some embodiments, a typical daily dosage of agonist of Mrgprs, for example MrgprX1 agonist, is from about 10 μM to about 1 mM, or from about 50 μM to about 0.8 mM, from about 100 μM to about 0.5 mM, from about 200 μM to about 400 μM, or from about 300 μM to about 350 μM.

Toxicity and therapeutic efficacy of an agonist of Mrgprs can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The agonists of Mrgprs exhibiting large therapeutic indices are preferred. While compounds that exhibit toxic side effects can be used, care can be taken to design a delivery system that targets such compounds to the site of affected tissue in order to minimize undesired side effects.

### Kits

The compositions disclosed herein, particularly compositions comprising MrgprX1 agonists that have been shown to treat or prevent persistent pain, and, preferably, do not impact perception of acute pain, may be assembled into kits or pharmaceutical systems for use in ameliorating, treating, preventing, or stopping persistent pain. Kits or pharmaceutical systems according to this aspect of the present disclosure comprise a carrier means, such as a box, carton, tube or the like, having in close confinement therein one or more container means, such as vials, tubes, ampules, bottles and the like. The kits or pharmaceutical systems disclosed herein may also comprise associated instructions for using the agents disclosed herein to treat persistent pain. The practice of the methods, compositions, kits or systems disclosed herein employs, unless otherwise indicated, conventional techniques, which are well within the purview of the skilled artisan.

### EXAMPLES

Some aspects of the embodiments discussed above are disclosed in further detail in the following examples, which are not in any way intended to limit the scope of the present disclosure.

### Experimental Methods

The following experimental methods were used for Examples 1-6 described below.

Vector Construction

To delete a cluster of Mrgpr genes in the mouse germ line, two replacement vectors were constructed for MrgprA 1 and MrgprB4, which reside on each end of the Mrgpr cluster, respectively. The genomic sequences of MrgprA 1 and MrgprB4 were obtained from the Mouse Genome Project (NCBI). The entire open reading frames (ORFs) of both MrgprA1 and MrgprB4 are encoded by a single exon. The arms of the MrgprA1 and MrgprB4 targeting constructs were obtained by PCR amplification from 129/SvJ genomic DNA using Expand High Fidelity PCR System (Roche).

The MrgprA1 targeting vector was constructed by inserting an eGFPf/IRES-rtTA/loxP/Ace-Cre/PGK-neomycin/loxP cassette between the 5′ and 3′ arms. For the MrgprB4 targeting vector, a PLAP/loxP/PGK-hygromycin cassette was cloned between the 5′ and 3′ arms.

MrgprA1 and MrgprB4 targeting vectors were electroporated into mouse CJ7 embryonic stem (ES) cells by two rounds of electroporation. Correct recombination at both loci was verified by PCR with genomic DNA of the clones using primer sets flanking the 5′ and 3′ arms of the targeting construct. This was further confirmed by Southern Blot hybridization using probes that flanked the 5′ arms of the targeting constructs. A third round of electroporation with CMV-Cre was conducted in an ES cell clone with both MrgprA1 and MrgprB4 loci correctly targeted. The deletion of genomic DNA between the two loci (845 kb) in the ES cells by Cre/loxP-mediated recombination was confirmed by PCR using primers flanking the two loci and Southern blot. Chimeric Mrgpr-clusterΔ mice were produced by blastocyst injection of positive ES cells. Mrgpr-clusterΔ^{+/-} mice were generated by mating chimeric mice to C57BI/6 mice.

Chronic Constriction Injury Model of Neuropathic Pain in Mice

A chronic constriction injury (CCI) at the left sciatic nerve was induced in adult male mice. Inhalation anesthesia was induced with a constant level of isoflurane (2.0%) delivered through a nose cone. Under aseptic conditions, the left sciatic nerve at the middle thigh level was separated from the surrounding tissue and loosely tied with three nylon sutures (9-0 nonabsorbable-monofilament, S&T AG, Neuhausen, Switzerland). The distance between two adjacent ligatures was around 0.5 mm. None of the mice displayed autotomy or exhibited marked motor deficits.

Behavioral Studies

All behavioral tests were performed with an experimenter blinded to the genotype. The mice used in the tests were backcrossed to C57BI/6 mice for at least five generations and were 2 to 3 month-old males (20-30 g).

Formalin test: formalin (5 μl of 2% formalin in phosphate-buffered saline) was injected into the plantar region of one hind paw, and spontaneous pain behavior (licking and biting) was recorded for 60 min as previously described in Chen et al., 2006, Neuroscience, 141:965-975; Knabl et al., 2008, Nature 451:330-334.

CFA-induced heat hyperalgesia: the intraplantar region of one hind paw of each mouse was injected with 6 μl of 50% CFA solution or 10 μl of 1% carrageneen solution in saline. Thermal pain sensitivity was assessed by recording paw withdrawal latencies on exposure to a defined radiant heat stimulus (Hargreaves test) before CFA injection and 30 min after injection.

Tail immersion test: mice were gently restrained in a 50-ml conical tube that the mice voluntarily entered. The protruding one third of the tail was then dipped into a 50° C. water bath. Latency to respond to the heat stimulus with vigorous flexion of the tail was measured three times and averaged.

CCI-induced mechanical allodynia: mechanical sensitivity was assessed with the von Frey test using the frequency method. Two calibrated von Frey monofilaments (low force: 0.07 g and high force: 0.45 g) were used. Each von Frey filament was applied perpendicularly to the plantar side of each hind paw for approximately 1 sec; the stimulation was repeated 10 times to both hind paws. The occurrence of paw withdrawal in each of these 10 trials was expressed as a percent response frequency: paw withdrawal frequency (PWF)=(number of paw withdrawals/10 trials)×100%.

Drug and Intrathecal Injection

BAM 8-22 was purchased from Tocris (Bristol, UK) and was suspended in 0.9% saline. The drug was injected intrathecally under brief isoflurane (1.5%) anesthesia to reduce stress. A 30-gauge, 0.5-inch needle connected to a 10-μl syringe was inserted into one side of the L5 or L6 spinous process at an angle of approximately 20° above the vertebral column and slipped into the groove between the spinous and transverse processes. The needle was moved carefully forward to the intervertebral space. A tail flick indicated that the tip of the needle was inserted into the subarachnoid space.

Electrophysiological Recording of Wide Dynamic Range (WDR) Neurons

The electrophysiological recording of WDR neurons in the dorsal horn of the spinal cord was performed by an experimenter blinded to the genotype as previously described in Guan et al., 2006, J. Neurosci., 26:4298-4307. Mice were paralyzed with pancuronium bromide (0.15 mg/kg, i.p.) during neurophysiological recording. Throughout the experiment, anesthesia was maintained with a constant level of isoflurane (1.5%) carried in med-air. A spinal unit with a cutaneous receptive field located in the plantar area of the hind paw was located by applying mechanical stimuli. WDR neurons were defined as those that responded to both innocuous and noxious mechanical stimuli and that had increasing rates of response to increasing intensities of stimuli. Electrical stimuli were applied through a pair of fine needles inserted subcutaneously across the central plantar area of the hind paw 0.3-0.4 cm apart. Extracellular recordings of individual neurons were obtained by using fine-tip (<1.0 μm) paralyn-coated tungsten micro-electrodes (8 mΩ at 1 kHz).

BAM 8-22 or vehicle control was applied directly to the exposed surface of the spinal cord at the recording segment in a volume of 30-50 μl following pre-drug tests. The effects of BAM 8-22 on the spontaneous activity of WDR neurons were examined within 0-10 min after application. The evoked neuronal responses were recorded 10-30 minutes after drug application. Only one neuron in each animal was used to test the drug effects. The post-drug responses were compared with the pre-drug responses, allowing each neuron to act as its own control.

Calcium Imaging of Cultured Dorsal Root Ganglia (DRG) Neurons

DRG were dissected from newborn or adult mice and dissociated as previously described in Caterina et al., 1999, Nature 398:436-441. DRG neurons were plated on poly-D-lysine/laminin-coated coverslips and cultured for 24 hours. Calcium imaging was performed as described in Caterina et al., 1997, Nature 389:816-824. Briefly, neurons were loaded with Fura 2-acetomethoxy ester (Molecular Probes) for 30 min in dark at room temperature. After washing, neurons were imaged at 340/380 nm excitation to detect intracellular free calcium. All tests were performed with an experimenter blinded to the genotype.

Whole-Cell Voltage-Clamp Recordings of Cultured DRG Neurons

Dissociated DRG neurons were plated on coverslips and cultured for 2-7 hours. For clamp recording, coverslips were perfused with extracellular solution (ECS) consisting of (in mM): NaCl 140, KCl 4, CaCl₂ 2, MgCl₂ 2, HEPES 10, Glucose 5, with pH adjusted to 7.38 using NaOH. Neurons with cell body diameters between 22 and 25 μm were recorded in the whole-cell voltage-clamp configuration with electrodes (pipette solution in mM: KCl 135, MgATP 3, Na₂ATP 0.5, CaCl₂ 1.1, EGTA 2, Glucose 5, pH adjusted to 7.38) using an Axon 700B amplifier and the pCLAMP 9.2 software package (Axon Instruments). Electrodes were pulled (Narishige, Model pp-830) from borosilicate glass (WPI, Inc), and after filling they had resistances of 2-4 MΩ. Neurons were perfused with 10 μm FMRFamide for 30 seconds and washed for 3 minutes. Action potentials were elicited with prolonged injections (500 ms) of a depolarizing current (200 pA) before and after FMRFamide treatment. All experiments were performed at room temperature with an experimenter blinded to the genotype.

Data Analysis

The number of action potentials (APs) evoked by graded electrical stimuli was compared between two genotypes, using a two-way mixed-model analysis of variance (ANOVA) with Fisher's protected least significant difference (LSD) post-hoc test. Student's t-test was used to compare the recording depth, activation threshold and latency of the first A-fiber-mediated and the first C-fiber-mediated responses, respectively, between the two groups. For wind-up, the raw data were the number of APs in the C-component evoked by each stimulus in a train of repetitive electrical stimulation. Since the number of APs in the C-component varies among WDR neurons, the raw data for each neuron were normalized to the first response in each trial (input) and then averaged. A two-way mixed-model ANOVA was used to compare wind-up and the averaged C-component responses to the last 10 stimuli (7^{th}-16^{th}) of the trial between the two genotypes and between pre- and post-drug conditions. Data are presented as mean±standard error of mean (S.E.M). Statistical comparisons were made using unpaired Student's t-test and differences were considered significant at p<0.05.

### Example 1

### Targeted Deletion of a Cluster of Mrgpr Genes in Mice

This example shows the generation of Mrgpr-clusterΔ^{-/-} mice in which a cluster of 12 intact Mrgpr genes was deleted.

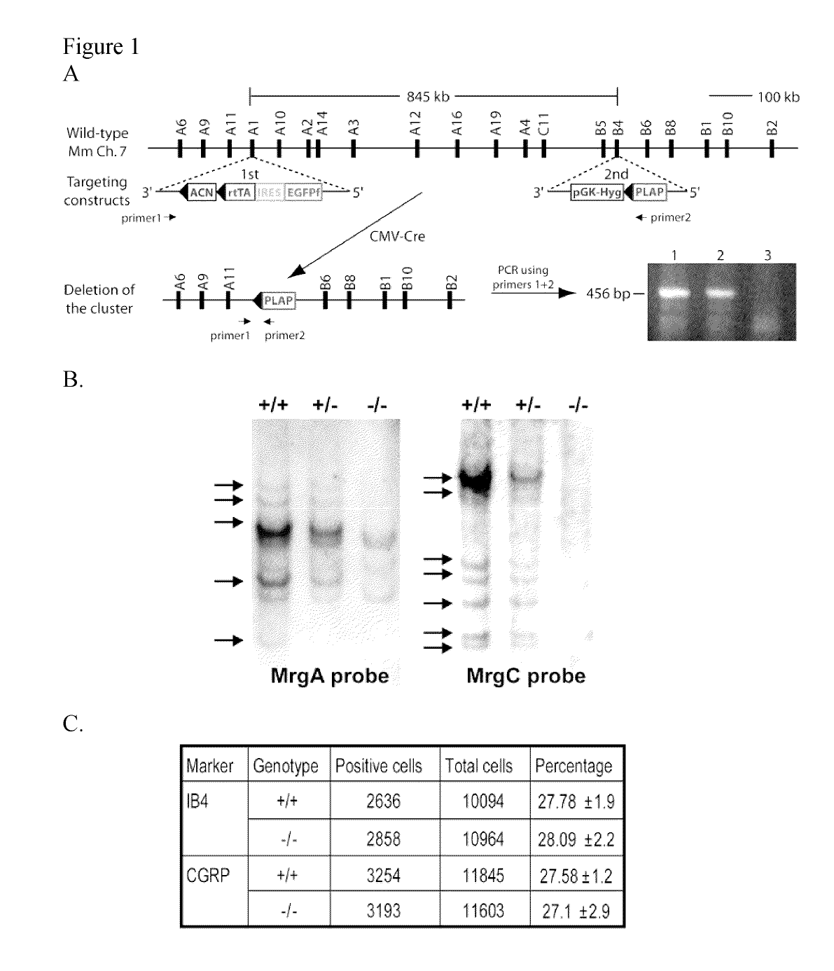

Many Mrgpr genes are clustered together on Chromosome 7 in mice. A mouse line in which a cluster of Mrgpr genes was deleted (FIGS. 1A-B) was generated. The deleted 845-kilobase region comprises ˜30 Mrgpr genes, 12 of which (MrgprA1-4, A10, A12, A14, A16, A19, B4, B5 and C11) have intact open reading frames (ORFs, FIG. 1A). No other ORF is present in this region, according to the Mouse Genome Project. The mouse Mrgpr superfamily consists of more than 50 members, and more than half of them are pseudogenes and only ˜24 genes have intact ORFs. Thus, the deleted cluster represents ˜50% of the potentially functional Mrgpr repertoire and contains most MrgprA and MrgprC genes as well as some members of the MrgprB subfamily. Most of the deleted Mrgpr genes are exclusively expressed in DRG. MrgprA6, A9, A11, B1, B2, B6, B8, B10, D-G are not included in this deletion based on the Mouse Genome Project and RT-PCR experiments. It was observed that MrgprB1 and Mrgpr82, which were not deleted, are expressed in the skin but not in DRG (Zylka et al., Proc. Natl. Acad. Sci. USA, 2003, 100:10043-10048).

Chimeric Mrgpr-clusterΔ^{-/-} mice were produced by blastocyst injection of positive embryonic stem cells. The Mrgpr-clusterΔ^{-/-} mice were generated by mating chimeric mice to C57Bl/6 mice. Mice were backcrossed to C57Bl/6 mice for at least five generations. Homozygous Mrgpr-clusterΔ^{-/-} mice were viable, fertile and generally indistinguishable from wild-type littermates in appearance, body weight, overt behavior and gross anatomy. The motor function of Mrgpr-clusterΔ^{-/-} mice was also normal as determined by the rotarod test. To determine if neuronal survival was compromised in the absence of the Mrgpr gene cluster, staining for NeuN (a pan-neuronal marker) was performed and NeuN cells in lumbar (L5) DRG was counted. The total number of L5 DRG neurons was found to be comparable between wild-type and Mrgpr-clusterΔ^{-/-} mice (15844±933 and 16396±1037, respectively, n=3), suggesting that Mrgprs were not required for the survival of primary sensory neurons

Mrgprs are specifically expressed in subsets of small-diameter primary sensory neurons in DRG (Dong et al., 2001; Zylka et al., 2003). Small-diameter sensory neurons can be broadly divided into two classes: peptidergic and nonpeptidergic (Hunt and Mantyh, 2001). Peptidergic neurons express the neuropeptides substance P and CGRP. Nonpeptidergic neurons do not express substance P but can be visualized with the lectin IB4 labeling. Most murine Mrgpr are expressed in the nonpeptidergic subclass (Dong et al., 2001; Zylka et al., 2003). To determine if Mrgprs are required for proper differentiation of DRG neurons, the proportion of these two subsets of neurons were measured. The proportion of these two subsets did not differ between wild-type and Mrgpr-clusterΔ^{-/-} mice (FIG. 1C), suggesting that Mrgprs are not required for fate determination or differentiation of small-diameter sensory neurons.

### Example 2

### Deletion of the Mrgpr Cluster Enhanced Inflammatory Pain Responses

This example shows that the deletion of Mrgpr genes in mice affected nocifensive behavioral responses to painful stimuli.

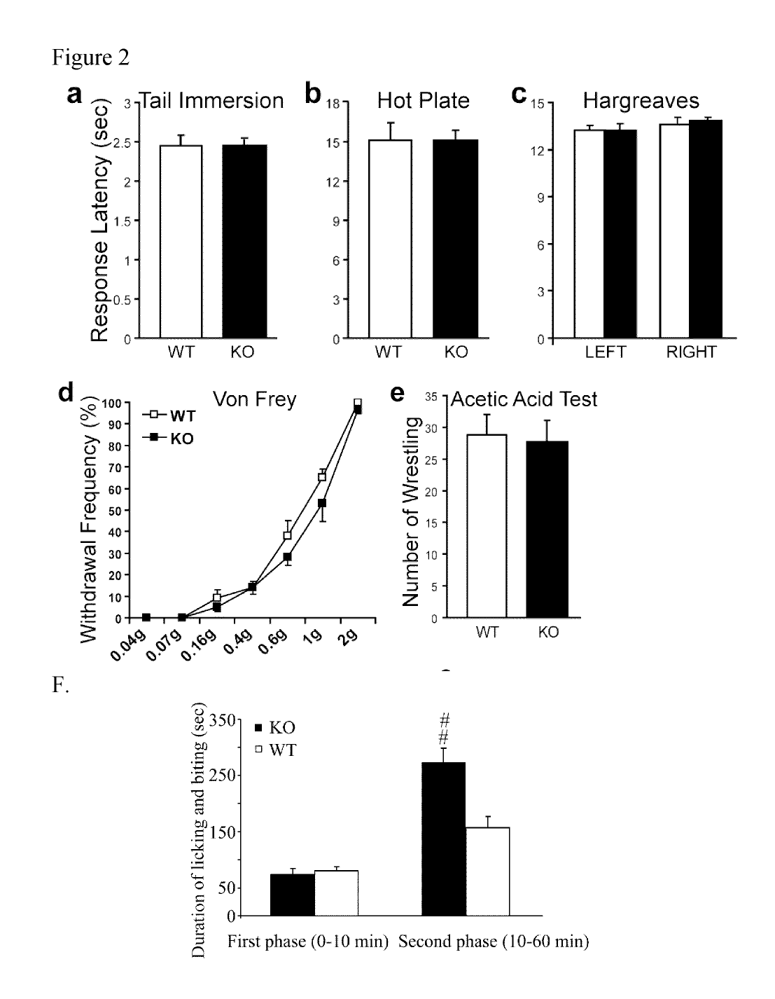

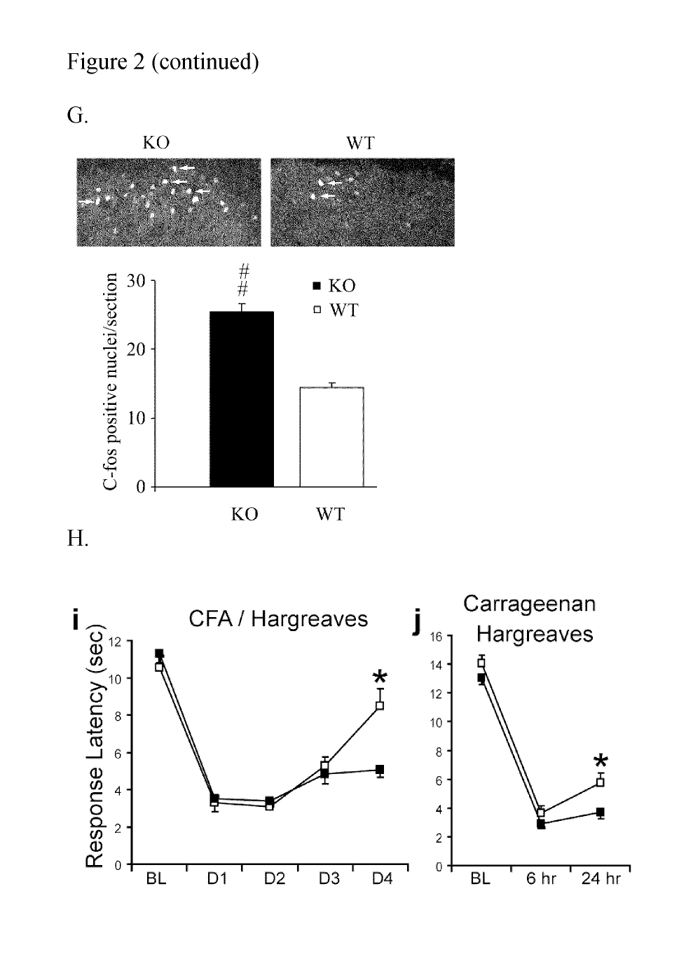

Mrgpr-clusterΔ^{-/-} mice was found to respond normally to acute noxious thermal, mechanical and chemical stimulation as compared with wild-type littermates (FIG. 2A-2E). It suggested that acute pain sensation was unaffected in Δ^{-/-} mice. However, in the inflammatory state, Mrgpr-clusterΔ^{-/-} mice displayed enhanced pain responses.

The formalin test, a unique model of persistent pain that encompasses inflammatory, neurogenic, and central mechanisms of nociception, was used to determine whether formalin-induced tissue injury leads to an endogenous activation of Mrgprs to modulate spontaneous pain. In the formalin test, spontaneous pain responses to two principally different stimuli, nociceptor activation (first phase) and tissue inflammation (second phase) can be readily revealed in the same test and separately analyzed.

Formalin was injected into the plantar tissue of one hind paw. It was observed that spontaneous pain behavior in the second phase (10-60 min post-injection), which is driven largely by tissue inflammation and involves central sensitization of dorsal horn neurons, was significant potentiated in the Mrgpr-clusterΔ^{-/-} mice, as compared with wild-type littermates, while the response in the first (acute) phase was unaffected (FIG. 2F). Reflecting this behavioral phenotype, a greater increase of c-fos-expressing neurons in the ipsilateral laminae I and II of lumbar (L4-L6) spinal segments in Mrgpr-clusterΔ^{-/-} mice than that in WT mice was observed after an intra-plantar injection of formalin (FIG. 2G). In contrast, the first (acute) phase of the formalin response (0-10 min post-injection), which resulted predominantly from a direct chemical stimulation of the C-fiber afferent nociceptors, was not affected by the mutation (FIG. 2F). These data indicated that inflammatory pain was enhanced in Mrgpr-clusterΔ^{-/-} mice.

Further, to determine the role of Mrgpr genes in inflammation-induced hyperalgesia and allodynia, mouse hindpaws were injected with complete Freund's Adjuvant (CFA), an agent that induces long term tissue inflammation associated thermal hyperalgesia and mechanical allodynia. During the first three days after injection, wild-type and Mrgpr-clusterΔ^{-/-} mice showed similar reductions in paw withdrawal thresholds to radiant heat stimuli, compared to pre-CFA thresholds. However, on the fourth day, wild-type mice began to recover from the thermal hyperalgesia, whereas the Mrgpr-clusterΔ^{-/-} mice remained in the hyperalgesic state (FIG. 2I). CFA also induced a marked reduction in the paw withdrawal threshold to punctate mechanical stimulation in Mrgpr-clusterΔ^{-/-} but not wild-type mice on the first day after injection (FIG. 2H). In another inflammatory model (carrageenen test), the paw withdrawal threshold to radiant heat stimuli was dramatically reduced in both wild-type and Mrgpr-clusterΔ^{-/-} mice 6 hours after injection. However, wild-type mice recovered from the hyperalgesia 24 hours after injection, whereas mutant mice did not show obvious recovery (FIG. 2J).

### Example 3

### Deletion of the Mrgpr Cluster Enhanced the Wind-Up Response of Dorsal Horn Wide Dynamic Range (WDR) Neurons

This example shows that the enhanced inflammatory pain response in mice with deletion in Mrgpr genes was also manifested at the cellular level of spinal pain processing.

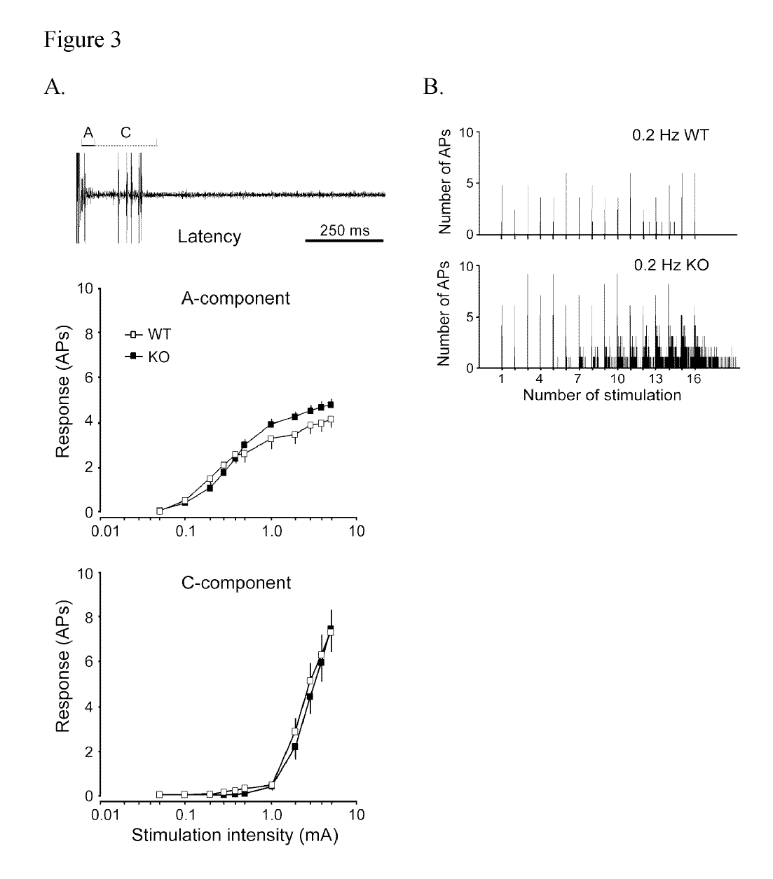

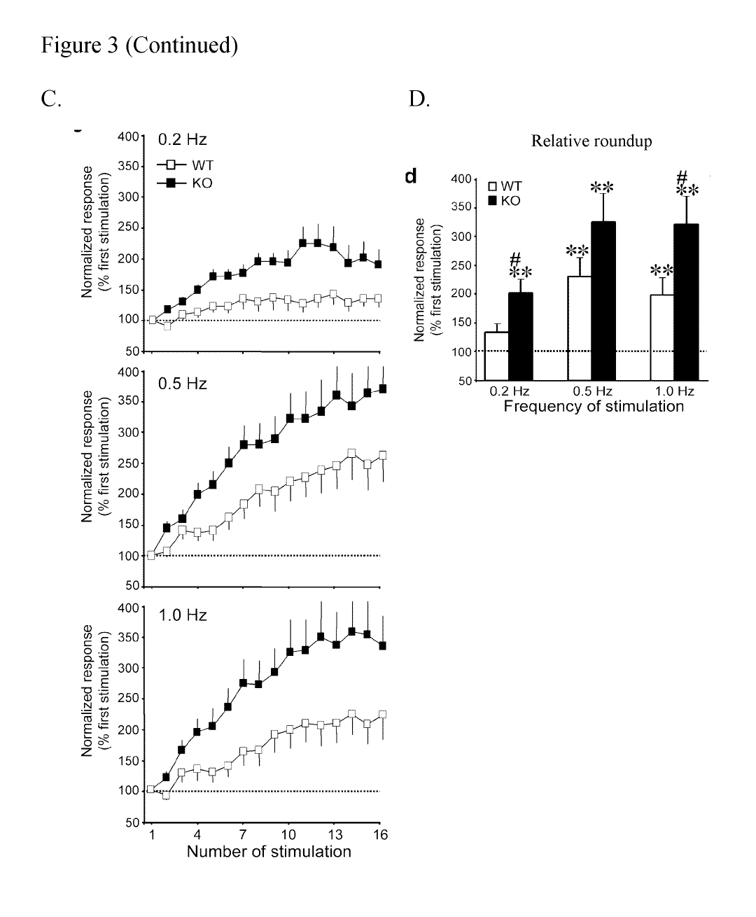

WDR neurons in the deep dorsal horn are important for spinal pain processing and are candidates for transmission-cells in the gate theory of pain. These neurons receive both innocuous and noxious sensory inputs from the periphery and display A-fiber- and C-fiber-mediated responses (A- and C-components, respectively) to a single intra-cutaneous electrical stimulus with an intensity above C-fiber activation threshold (FIG. 3A). Based on the axon conduction velocities, WDR neuronal responses to electrical stimuli in mice were separated into a short latency A-component (0-40 msec, excluding stimulus artifact) and a long latency C-component (40-250 msec). Typically, the excitability of some WDR neurons progressively increases in response to repetitive C-fiber afferent stimulation, a short-term activity-dependent neuronal sensitization called “windup.”

To determine whether the enhanced inflammatory pain was manifested at the level of nociceptive processing in the CNS in Mrgpr-clusterΔ^{-/-} mice, the excitability of WDR neurons within the spinal dorsal horn that receive both innocuous and noxious sensory input from the periphery were recorded. Windup was examined by repetitive intra-cutaneous electrical stimuli (16 pulses, 3.0 mA/supra C-fiber activation threshold, 2.0 msec) applied at 0.2 Hz and 1.0 Hz, and with a minimum 10-min interval between each trial. It was observed that WDR neurons in WT mice (n=23) showed windup responses at the higher frequency of 1.0 Hz stimulation, but rarely at 0.2 Hz stimulation (FIGS. 3B-D). The averaged C-component responses to the last stimuli (7^{th}-16^{th}) of the trial (wind-up values) were significantly increased during 0.5 and 1.0 Hz, but not 0.2 Hz, stimulation in wild-type mice as compared to the baseline input (response of WDR neurons to the first stimulus of each trial, FIG. 3D). In contrast, many WDR neurons in Mrgpr-clusterΔ^{-/-} mice (n=30) exhibited wind-up responses to 0.2 Hz stimulation (FIGS. 3B and 3D), and wind-up values were significantly higher than baseline (FIG. 3D). Also, the wind-up responses to both 0.2 Hz and 1.0 Hz stimulation were significantly higher in Mrgpr-clusterΔ^{-/-} mice than in WT mice (p<0.05, FIGS. 3C and 3D). As wind-up mimics some characteristic features of spinal sensitization in the inflammatory state, the potentiated wind-up observed in Mrgpr-clusterΔ^{-/-} mice correlated well with the enhanced inflammatory pain behavioral phenotype.

To quantify the peak levels of windup, the relative windup value (that is, the averaged C-component responses to the last 10 (7^{th}-16^{th}) stimuli of the trial, normalized by the C-component response to the first stimulus of each trial (input value)) was also measured. The relative windup values were significantly increased during 1.0 Hz, but not 0.2 Hz, stimulation in WT mice, as compared to the respective baseline (FIG. 3E). Also, unlike the case in WT mice, many WDR neurons in Mrgpr-clusterΔ^{-/-} mice (n=30) exhibited windup at the normally ineffective 0.2 Hz stimulation frequency (FIGS. 3B and 3D), and the relative windup value at 0.2 Hz stimulation in the Mrgpr-clusterΔ^{-/-} group was significantly greater than the baseline (FIG. 3E). Importantly, the windup responses at both 0.2 Hz and 1.0 Hz stimulation frequencies were significantly greater in Mrgpr-clusterΔ^{-/-} than in WT mice (P<0.05, FIG. 3D). The mean recording depth of WDR neurons did not differ between WT (450±23 μm) and Mrgpr-clusterΔ^{-/-} mice (441±22 μm, P>0.05). In contrast to their differences in windup, the acute responses of WDR neurons to graded intra-cutaneous electrical stimulation (0.05-5.0 mA, 2.0 msec) were comparable between the WT and Mrgpr-clusterΔ^{-/-} mice. The threshold and the population stimulus intensity-response (S-R) function of the C-component were also similar in the two groups (FIG. 3D, Table 1). These data suggest that one or more Mrgprs within the Mrgpr cluster function to limit the extent of increased WDR neuronal excitability in response to repetitive C-fiber stimulation.

TABLE 1
The activation thresholds and latencies of the first A-fiber mediated
and C-fiber-mediated responses of WDR neurons

| | A-latency | C-latency | A-threshold | C-threshold |
|---|---|---|---|---|
| | (msec) | (msec) | (mA) | (mA) |
| Wild-type | 14.0 ± 0.8 | 124 ± 5 | 0.23 ± 0.04 | 2.0 ± 0.3 |
| Mrgpr-clusterΔ^{-/-} | 14.8 ± 0.6 | 108 ± 6 | 0.26 ± 0.03 | 2.3 ± 0.2 |
| Wild-type | 14.2 ± 0.8 | 114 ± 5 | 0.16 ± 0.02 | 1.5 ± 0.3 |
| pre-BAM 8-22 | | | | |
| Wild-type | 12.1 ± 0.7 | 115 ± 4 | 0.17 ± 0.03 | 1.7 ± 0.4 |
| post-BAM 8-22 | | | | |
| Mrgpr-clusterΔ^{-/-} | 14.5 ± 0.6 | 125 ± 3 | 0.17 ± 0.02 | 1.7 ± 0.5 |
| pre-BAM 8-22 | | | | |
| Mrgpr-clusterΔ^{-/-} | 12.9 ± 0.6 | 118 ± 4 | 0.19 ± 0.03 | 1.9 ± 0.6 |
| post-BAM 8-22 | | | | |

* Data are expressed as mean ± SM.

### Example 4

### The Agonists for Mrgpr Receptors

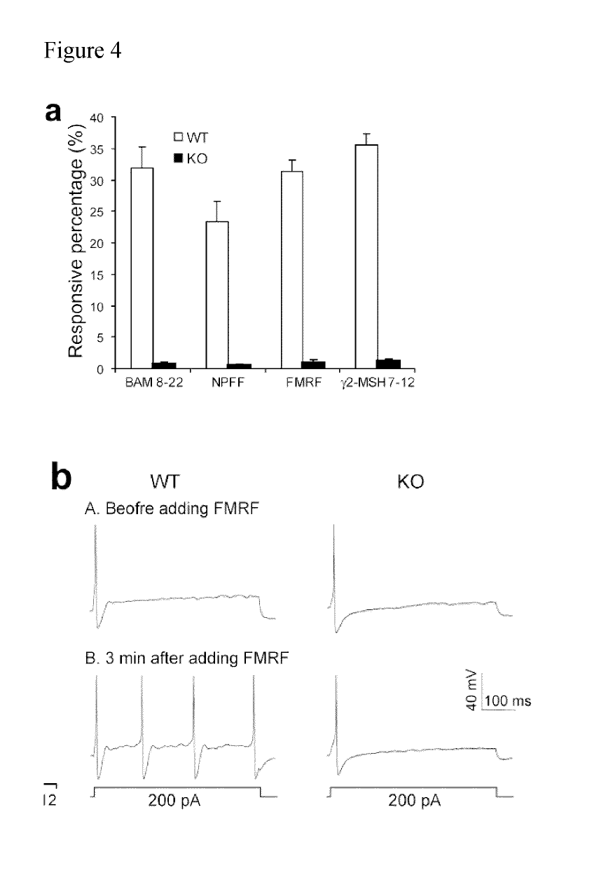

To determine if Mrgprs are potential receptors for RF-amide related peptides in the peripheral nervous system, the effect of RF-amide related peptides on DRG neurons from WT and Mrgpr-clusterΔ^{-/-} mice was studied. Calcium imaging indicated that BAM 8-22, γ2-MSH 7-12, NPFF and FMRFamide excited 23.38% to 35.55% of total DRG neurons from wild-type newborn mice, while these peptides triggered few or no responses in DRG neurons from Mrgpr-clusterΔ^{-/-} mice (FIG. 4A). In adult wild-type DRG neurons, these peptides evoked fewer responses ranging from 5.01% to 8.01%, consistent with Mrgpr expression patterns at different developmental stages. Adult mutant DRG neurons exhibited no responses.

To determine whether RF-amide related peptides function as modulators of neuronal excitability, electrophysiological analysis of WT and Mrgpr-clusterΔ^{-/-} neurons were carried out in cultures of dissociated adult DRG sensory neurons, using the peptide FMRFamide as an Mrgpr agonist. From current-clamp recordings, it was found that FMRFamide treatment of WT DRG neurons often caused a significant increase in the number of action potentials evoked by injection of depolarizing current (FIG. 4B). Such sensitizing effects were seen in a large portion of recorded WT DRG neurons with cell body diameters ranging from 22 to 25 μm (10 of 23 cells recorded), indicating that FMRFamide increases excitability in WT neurons (FIG. 4B). But this effect was rarely seen in neurons isolated from Mrgpr-clusterΔ^{-/-} mice (1 of 20 cells). The resting electro physiological properties of DRG neurons were not altered by Mrgpr deletion. Resting membrane potentials (−57.0±1.0 vs. −57.7±1.6 mV, n=20) and cell capacitance (12.9±0.4 vs. 13.4±0.5 pF, n=20) were similar between WT and mutant neurons. These data demonstrate that FMRFamide, an Mrgpr agonist, positively regulates neuronal excitability in an Mrgpr-dependent manner.

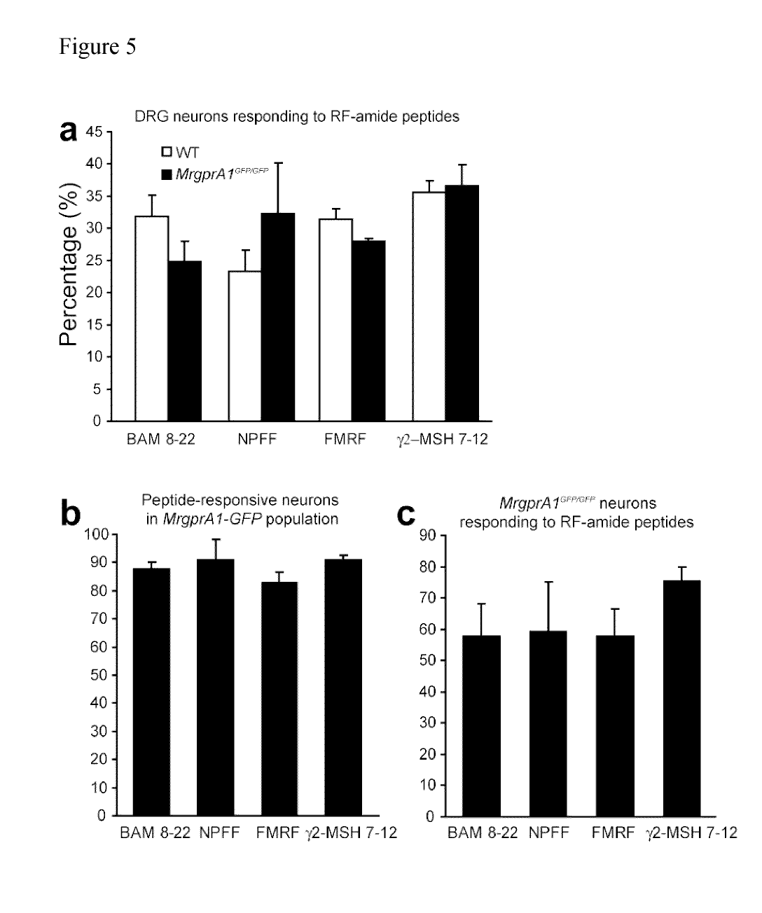

Calcium imaging and electrophysiological data suggested that Mrgprs were necessary for functional responses to RF-amide related peptides in DRG neurons, and may be the exclusive receptors for these peptides. But, the percentage of DRG neurons responding to these peptides did not differ significantly between WT and homozygous MrgprA1^{GFP/GFP} mice in which the coding sequence of MrgprA1 was replaced with an in-frame fusion of GFP (FIG. 5A), suggesting that the effects of these peptides were not mediated by MrgprA1 itself. Approximately 90% of RF-amide-responsive neurons were found within the MrgprA1⁺ population (FIG. 5B). These data implied that other Mrgprs co-expressed with MrgprA1 (such as MrgprC11) may mediate responses to these peptides.

### Example 5

### Intrathecal Administration of BAM 8-22 Inhibited Persistent Inflammatory Pain and Neuropathic Pain in WT, But not in Mrgpr-clusterΔ^{-/-} Mice

In this example, the effects of intrathecal BAM 8-22 on pain behavior in WT and Mrgpr-clusterΔ^{-/-} mice were examined.

Intrathecal injection of BAM 8-22 in awake mice elicited short-term (less than 10 min) spontaneous pain responses. The same response was observed in both WT and Mrgpr-clusterΔ^{-/-} mice, suggesting that it was Mrgpr-independent.

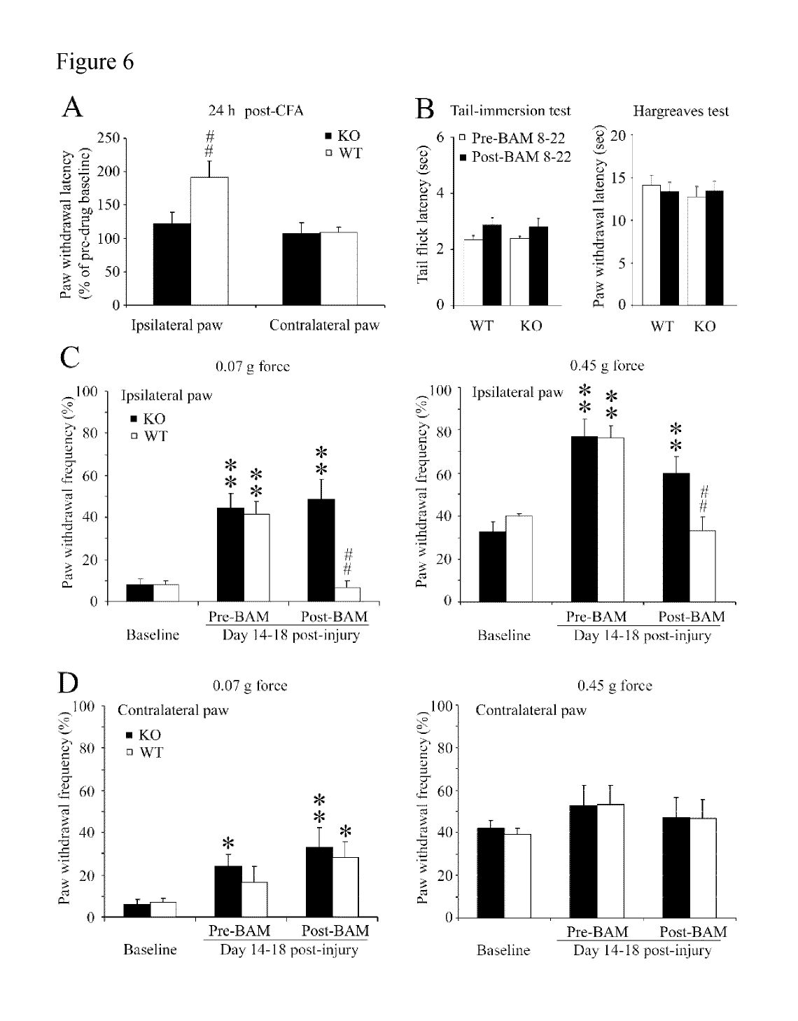

The ability of BAM 8-22 to modulate persistent inflammatory pain was determined by examining enhanced pain sensitivity to a noxious heat stimulus, as monitored by the Hargreaves test 24 hours after intra-plantar injection of CFA (6 μl, 50%) into one hind paw. In the absence of BAM 8-22, thermal hyperalgesia was comparable between the two genotypes: the paw-withdrawal latencies (PWL) of the ipsilateral hind paw in Mrgpr-clusterΔ^{-/-} and WT mice were 4.3±0.7 sec and 3.3±0.3 sec, respectively. In WT mice, a single intrathecal injection of BAM 8-22 (1 mM, 5 μl) was able to alleviate thermal hyperalgesia at 30 min post-injection, increasing the PWL by 1.9-fold compared to pre-drug baseline (FIG. 6A, WT, n=13, ipsilateral paw). In contrast, this anti-hyperalgesic effect of BAM 8-22 was not observed in Mrgpr-clusterΔ^{-/-} mice (FIG. 6A, Mrgpr-clusterΔ^{-/-}, n=10), indicating that the effect was Mrgpr-dependent. In addition, intrathecal BAM 8-22 did not significantly affect the PWL of the contralateral (control side) hind paw to acute radiant heat in either group (FIG. 6A, contralateral paw). These data suggest that Mrgprs (most likely MrgprC11) played a role in mediating the effect of exogenously administered BAM 8-22 to attenuate inflammatory thermal hyperalgesia in WT mice, and BAM 8-22 could function as an anti-hyperalgesic gent in vivo.

To determine whether intrathecal BAM 8-22 also affected acute thermal nociception, tail-immersion tests in naïve WT and Mrgpr-clusterΔ^{-/-} mice were performed. The tail flick latencies after intrathecal injection of BAM 8-22 were not significantly different from the respective pre-drug values in either WT (n=10) or Mrgpr-clusterΔ^{-/-} mice (n=10, FIG. 6B). This finding is in line with the observation that the peptide did not significantly change the sensitivities to acute radiant heat (Hargreaves test) in WT (n=15) and Mrgpr-clusterΔ^{-/-} (n=14, FIG. 6B) mice. In addition, thermal sensitivities were comparable between the two groups before and after BAM 8-22 treatment (FIG. 6B).

To determine whether BAM 8-22 could also reduce neuropathic mechanical allodynia in mice, mice were subjected to the chronic constriction injury (CCI) model, in which the sciatic nerve is ligated with a suture. The effect of this manipulation on mechanical pain sensitivity was tested at 14-18 days after injury, by measuring paw withdrawal frequency to punctate mechanical stimuli of different strengths. Intrathecal injection of BAM 8-22 (0.5 mM, 5 μl) significantly attenuated CCI-induced mechanical pain hypersensitivity to both low-force (0.07 g) and high-force (0.45 g) stimuli applied to the ipsilateral hind paw in WT mice (FIG. 6C, n=7). This anti-hyperalgesic effect of the peptide was eliminated in Mrgpr-clusterΔ^{-/-} mice (FIG. 6C, n=8), although the development of mechanical allodynia itself was not affected by the mutation. BAM 8-22 did not significantly change paw withdrawal responses on the uninjured side (FIG. 6D, contralateral paw). These results suggested that the anti-allodynic effect of intrathecal BAM 8-22 under neuropathic conditions was also mediated by Mrgprs.

### Example 6

### Spinal Application of BAM 8-22 Attenuated Wind-Up in WT Mice, But not in Mrgpr-clusterΔ^{-/-} Mice

This example shows that BAM 8-22, an MrgprC11 agonist, attenuated spinal neuronal sensitization involved in persistent pain.

To determine whether the anti-hyperalgesic effect of BAM 8-22 can be seen at the level of central nociceptive processing, the effects of spinal topical application of BAM 8-22 on the wind-up responses of WDR neurons to repetitive noxious inputs were determined.

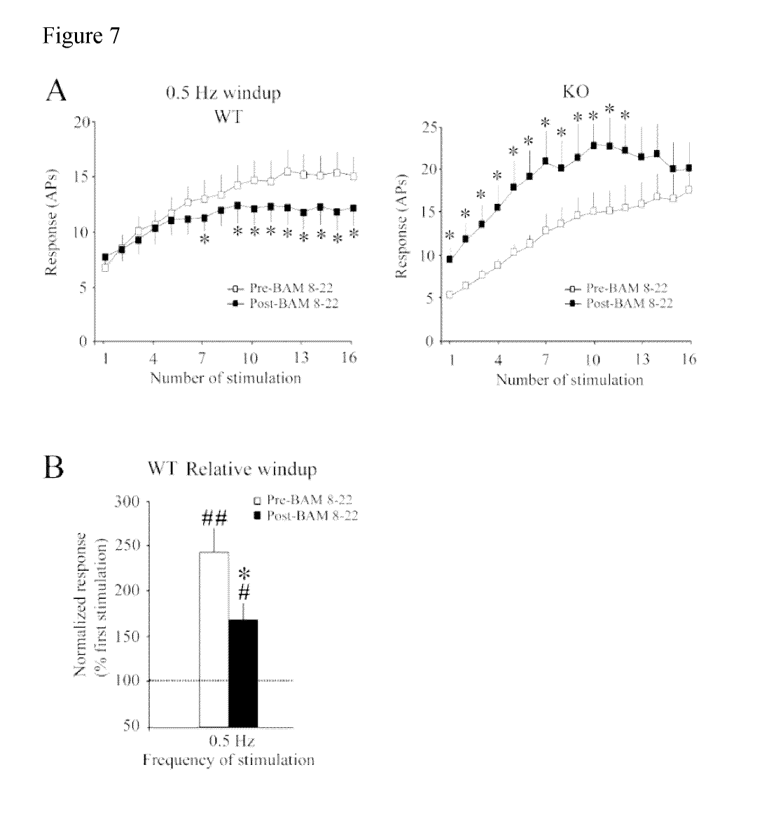

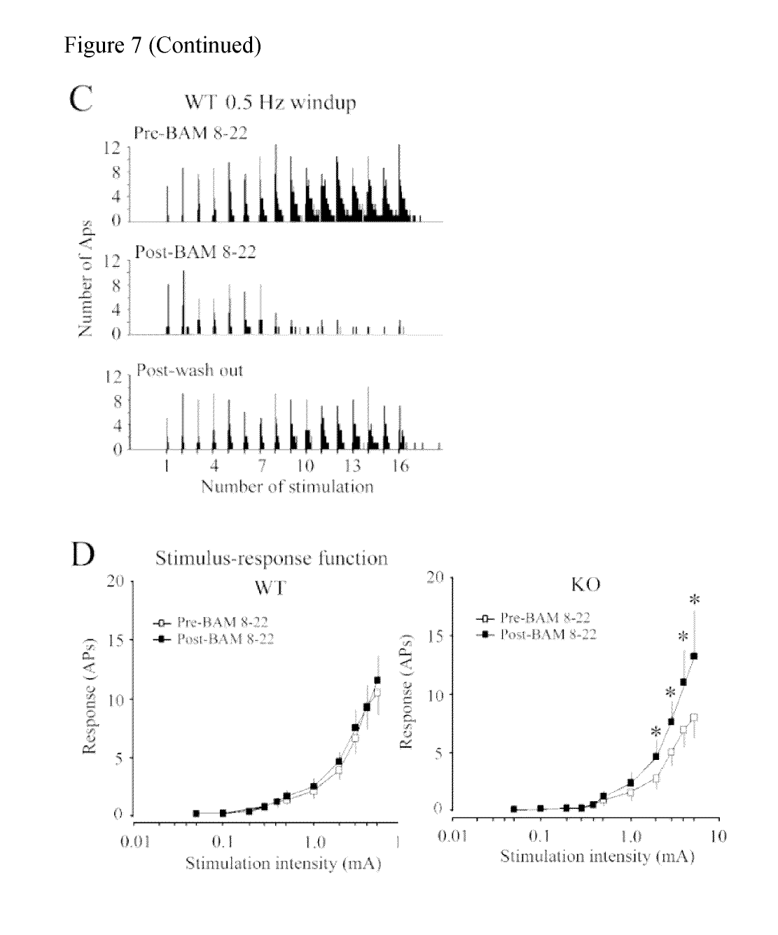

0.5 Hz stimulation frequency was used to induce windup without saturating the response. In WT mice (n=25), windup to 0.5 Hz stimulation was significantly attenuated after spinal superfusion with BAM 8-22 (FIG. 7A-C, WT), consistent with the anti-hyperalgesic effect of BAM 8-22 in the behavioral studies disclosed above. In Mrgpr-clusterΔ^{-/-} mice, by contrast, the effect of BAM 8-22 was not simply eliminated, but rather reversed: the peptide significantly increased the input value and C-component responses of WDR neurons to 0.5 Hz stimulation (FIG. 7A). Population S-R functions of C-fiber-mediated responses of WDR neurons to graded electrical stimulation were similar before and after drug administration in WT mice (n=25, FIG. 7D). However in Mrgpr-clusterΔ^{-/-} mice, BAM 8-22 significantly increased the number of C-component responses to graded electrical stimulation at intensities >1.0 mA (n=17, FIG. 7D). Nevertheless, the threshold and latency of the first C-fiber-mediated action potential was unaffected by administration of the peptide in either WT or Mrgpr-clusterΔ^{-/-} mice (Table 1). Saline, used as a control for BAM 8-22, did not affect the response properties of WDR neurons studied. These results suggested that the inhibitory effects of BAM 8-22 on windup, a measure of short-term neuronal hyperexcitability, were mediated by Mrgprs. Furthermore, deletion of Mrgprs unmasked a potentiating effect of the peptide on the same response, mediated by another class of receptors.

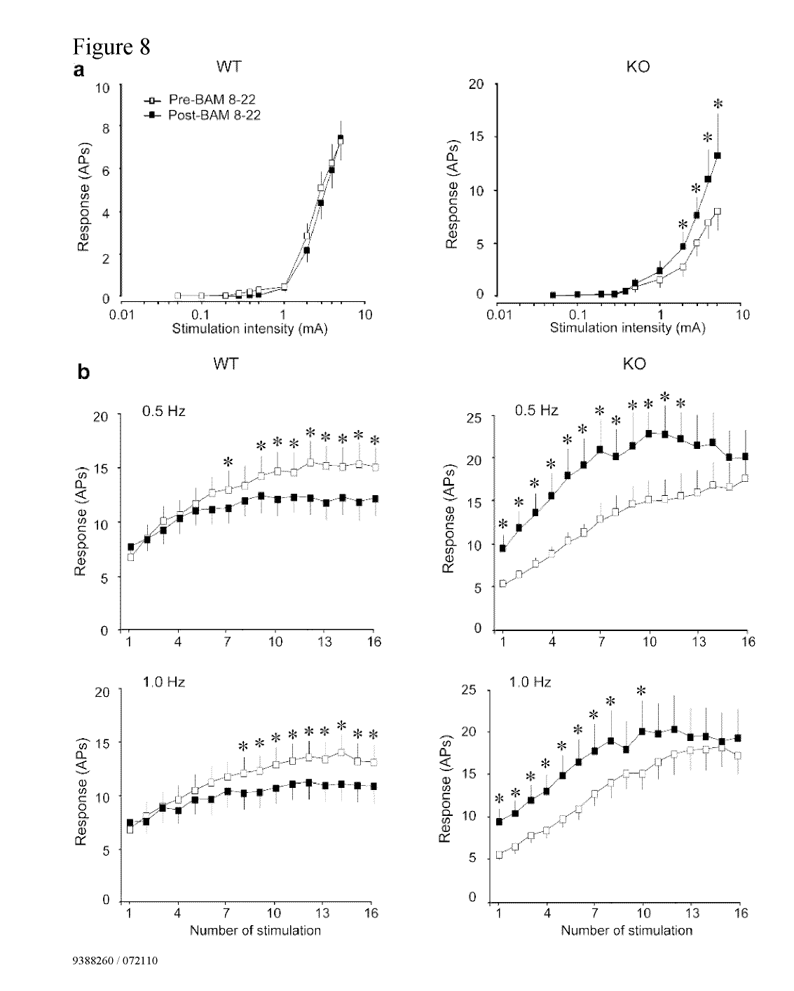

In WT mice (n=25), wind-up responses to both 0.5 Hz and 1.0 Hz stimulation were significantly attenuated after spinal superfusion with BAM 8-22 (0.1 mM, 30-50 μl, FIG. 8B), consistent with the anti-hyperalgesic effect of BAM 8-22 on nociceptive behaviors. In Mrgpr-clusterΔ^{-/-} mice, by contrast, BAM 8-22 significantly increased the input value and C-component responses of WDR neurons to repetitive stimulation (FIG. 8B). However the wind-up levels (normalized to the input value) were not increased, due to the increased input value. These results reveal a stimulatory effect of BAM 8-22 on spinal sensitivity in the absence of the Mrgpr cluster, supporting the idea that the short-term spontaneous pain induced by intrathecal infusion of this peptide is Mrgpr-independent, while the longer-term inhibitory effect is mediated by these receptors.

### Example 7

### Identification of MrgprX1 Agonists

This example illustrates the identification of MrgprX1 agonists.

Compounds to be tested for the potential to be effective for activating MrgprX1 are provided. As discussed above, the compounds may be, without limitation, small molecules (including both organic and inorganic molecules), peptides, peptide mimetics, nucleic acids, or antibodies.

In some embodiments, the compounds are initially screened for their ability to interact with MrgprX1. The candidate MrgprX1 agonist that binds to MrgprX1 is then administered to mammalian cells, such as HEK293 cells, stably expressing MrgprX1 gene in an intracellular calcium mobilization assay with the fluorometric imaging plate reader (FLIPR, Molecular Devices). The cells are monitored and measured for level of cell fluorescence, which indicates the extent of activation of MrgprX1 receptor. A successful MrgprX1 agonist is able to induce cell fluorescence to a level substantially comparable or higher in comparison to cells that is exposed to a control known MrgprX1 agonist (for example, BAM 8-22).

In some embodiments, the compounds are tested for their ability to modulate the level of MrgprX1 gene expression, preferably increasing the level of transcription of MrgprX1 gene. The level of transcription of MrgprX1 gene can be determined by measuring the level of MrgprX1 mRNA or MrgprX1 protein. The preferred MrgprX1 agonists significantly increase the level of MrgprX1 gene expression. In some embodiments, compounds are tested for their ability to enhance the level of MrgprX1 protein in cells. The level of MrgprX1 protein in cells can be determined by conventional techniques such as western blot. The preferred MrgprX1 agonists significantly increase the level of MrgprX1 protein in cells.

In some embodiments, the compounds are tested for their ability to positively allosterically modulate the activation of MrgprX1. The compounds are initially screened for their ability to interact with MrgprX1. A known MrgprX1 agonist (for example BAM 8-22) is administered to mammalian cells, such as HEK293 cells, stably expressing MrgprX1 gene in a low concentration. The cells are monitored and measured for level of cell fluorescence, which indicates the extent of activation of MrgprX1 receptor. The candidate MrgprX1 agonist that binds to MrgprX1 is then added to the cells in the presence of the low concentration of known MrgprX1 and tested for its positive allosteric modulation using a concentration-response (C/R) curve method. A successful MrgprX1 agonist acting as a positive allosteric modulator is able to significantly increase the amount of cell fluorescence triggered by the binding of low concentration of known MrgprX1 agonist (such as BAM 8-22) to MrgprX1 receptor.

### Example 8

### Identification of Therapeutics for the Treatment of Persistent Pain

This example illustrates the identification of compounds that can be used to treat, prevent, or ameliorate persistent pain.

Compounds to be tested for effective therapeutics for persistent pain are provided. As discussed above, the compounds can be, without limitation, small molecules (including both organic and inorganic molecules), peptides, peptide mimetics, nucleic acids, or antibodies. In some embodiments, the compounds are initially screened for their ability to activate Mrgprs, such as MrgprX1. Compounds that are able to activate Mrgprs can then tested for their ability to protect body from persistent pain.

### Example 9

### Treatment of Persistence Pain

This example illustrate the treatment of a patient suffering from or at risk of developing persistence pain, such as inflammatory and neuropathic pain.

A patient suffering from or at risk of developing persistence pain is identified and administered an effective amount of a pharmaceutical composition comprising one or more agonists of Mrgprs, for example one or more MrgprX1. A typical daily dose for an agonist of Mrgprs can range from about 0.01 μg/kg to about 1 mg/kg of patient body weight or more per day, depending on the factors mentioned above, preferably about 10 μg/kg/day to about 100 μg/kg/day. The appropriate dosage and treatment regimen can be readily determined by one of ordinary skill in the art based on a number of factors including the nature of the agonist of Mrgprs used, the route of administration and the patient's disease state. Treatment efficacy is evaluated by observing delay or slowing of disease progression, amelioration or palliation of the disease state, and/or remission.

The foregoing description and examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof. Although the present application has been described in detail above, it will be understood by one of ordinary skill in the art that various modifications can be made without departing from the spirit of the invention.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

All references cited herein including, but not limited to, published and unpublished patent applications, patents, text books, literature references, and the like, to the extent that they are not already, are hereby incorporated by reference in their entirety. To the extent that one or more of the incorporated literature and similar materials differ from or contradict the disclosure contained in the specification, including but not limited to defined terms, term usage, described techniques, or the like, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

What is claimed is:

1. A method of identifying compounds that reduce persistent pain in a mammal, comprising:
providing one or more MrgprX1 agonists;
testing the MrgprX1 agonists for their ability to reduce persistent pain;
testing the MrgprX1 agonists for their ability to modulate acute pain; and
identifying the MrgprX1 agonists that have the ability to reduce persistent pain but do not significantly modulate acute pain.

2. The method of claim 1, wherein testing for the ability to reduce persistent pain is carried out in an animal model of persistent pain.

3. The method of claim 2, wherein the animal model is a chronic constriction injury model of neuropathic pain.

4. The method of claim 1, wherein testing for the ability to modulate acute pain is carried out in an animal model of acute pain.

5. The method of claim 1, wherein said MrgprX1 agonists are small molecules, peptides, or nucleic acids.

6. The method of claim 1, wherein testing the MrgprX1 agonists for their ability to reduce persistent pain comprises administering the MrgprX1 agonists directly into spinal cord of an animal.

7. A method of treating persistent pain in a subject, comprising:
identifying a subject suffering from persistent pain; and
administering to the subject an effective amount of an MrgprX1 agonist.

8. The method of claim 7, additionally comprising the step of identifying an MrgprX1 agonist that reduces persistent pain but does not significantly change the perception of acute pain.

9. The method of claim 8, wherein the ability of the agonist to reduce persistent pain is measured in an animal model of persistent pain.

10. The method of claim 8, wherein the ability of the agonist to change the perception of acute pain is measured in an animal model of acute pain.

11. The method of claim 7, wherein said persistent pain is caused by inflammation.

12. The method of claim 7, wherein said persistent pain is caused by nerve injury.

13. The method of claim 7, wherein said MrgprX1 agonist binds to MrgprX1.

14. The method of claim 13, wherein said MrgprX1 agonist is a positive allosteric modulator of a ligand of MrgprX1.

15. The method of claim 14, wherein said ligand of MrgprX1 is selected from the group consisting of Bovine adrenal medulla 22 (BAM 22), BAM8-22, and P60 peptide.

16. The method of claim 7, additionally comprising administering a second MrgprX1 agonist.

17. The method of claim 16, wherein said MrgprX1 agonist activates MrgprX1 by enhancing the activity of the second MrgprX1 agonist.

18. The method of claim 7, where said MrgprX1 agonist is a small molecule, a peptide or a nucleic acid.

19. The method of claim 18, wherein said MrgprX1 agonist is a small molecule.

20. The method of claim 19, wherein said small molecule is selected from the group consisting of N-[3-(5-Chloro-6-oxo-4-piperazin-1-yl-6H-pyridazin-1-ylmethyl)-2-methyl-phenyl]-4-(6-methoxy-pyridin-3-yl)-benzamide, N-[3-(6-oxo-4-Piperazin-1-yl-6H-pyridazin-1-ylmethyl)-2-methylphenyl]-4-(6-methoxy-pyridin-3-yl)-benzamide, and

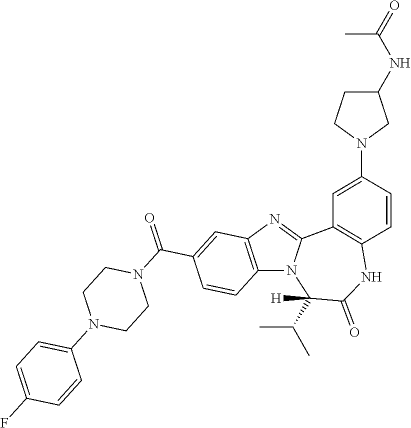

21. The method of claim 18, wherein said MrgprX1 agonist is a peptide.

22. The method of claim 21, wherein said peptide is selected from the group consisting of BAM 22, BAM 8-22, and P60 peptide.

23. The method of claim 7, wherein the subject is human.

24. The method claim 7, wherein said MrgprX1 agonist is delivered directed into the spinal cord of the subject.
